(19) United States
(12) Patent Application Publication
(10) Pub. No.: US 2026/0165599 A1
(43) Pub. Date: Jun. 18, 2026

(54) METHOD FOR CONTROLLING WEARABLE DEVICE, AND ELECTRONIC DEVICE FOR PERFORMING METHOD

(71) Applicant: Samsung Electronics Co., Ltd., Suwon-si (KR)

(72) Inventors: Wonkyung JANG, Suwon-si Gyeonggi-do (KR); Joayoung LEE, Suwon-si Gyeonggi-do (KR)

(21) Appl. No.: 19/427,965

(22) Filed: Dec. 19, 2025

Related U.S. Application Data
(63) Continuation of application No. PCT/KR2024/007078, filed on May 24, 2024.

(30) Foreign Application Priority Data
Jun. 30, 2023 (KR) 10-2023-0085152

(51) Int. Cl. A61B 5/08; A61B 5/00; A61B 5/0533; A61B 5/107; A61B 5/11; A61B 5/117; A61B 5/24; A61B 5/296

(52) U.S. Cl. CPC A61B 5/08; A61B 5/0002; A61B 5/0022; A61B 5/0533; A61B 5/1071; A61B 5/112; A61B 5/117; A61B 5/24; A61B 5/296; A61B 5/4514; A61B 5/6804; A61B 5/6807

(57) ABSTRACT

A wearable device control method may include: receiving, from a wearable device, first joint angle information about a first joint while the wearable device is controlled on the basis of a bicycle exercise program; determining a first joint angle of the first joint on the basis of the first joint angle information; determining whether the first joint angle corresponds to a first target joint angle; if the first joint angle corresponds to the first target joint angle, determining, on the basis of the bicycle exercise program, a first value of torque provided to the first joint; and controlling the wearable device on the basis of the first value of the torque.

### CROSS-REFERENCE TO RELATED APPLICATIONS

This application is a continuation application of International Application No. PCT/KR2024/007078, filed on May 24, 2024, in the Korean Intellectual Property Receiving Office, and claiming priority to KR Application No. 10-2023-0085152 filed Jun. 30, 2023, the disclosures of which are all hereby incorporated by reference herein in their entireties.

### TECHNICAL FIELD

Certain example embodiments may relate to a technology for controlling a wearable device.

### BACKGROUND

A change into aging societies has contributed to a growing number of people who experience inconvenience and pain from reduced muscular strength or joint problems due to aging. Thus, there is a growing interest in walking assist devices that enable elderly users or patients with reduced muscular strength or joint problems to walk with less effort and/or exercise.

### SUMMARY

A method of controlling a wearable device performed by an electronic device according to an example embodiment may include receiving first joint angle information on a first joint from the wearable device while the wearable device is controlled based on a bicycle exercise program, determining a first joint angle of the first joint based on the first joint angle information, determining whether the first joint angle corresponds to a first target joint angle, determining a first value of a torque provided to the first joint based on the bicycle exercise program when the first joint angle corresponds to the first target joint angle, and controlling the wearable device based on the first value of the torque.

An electronic device according to an example embodiment may include a communication module, comprising communication circuitry, configured to exchange data with an external device, and at least one processor, comprising processing circuitry, connected, directly or indirectly, to the communication module, and the processor(s) may perform receiving first joint angle information on a first joint from the wearable device while the wearable device is controlled based on a bicycle exercise program, determining a first joint angle of the first joint based on the first joint angle information, determining whether the first joint angle corresponds to a first target joint angle, determining a first value of a torque provided to the first joint based on the bicycle exercise program when the first joint angle corresponds to the first target joint angle, and controlling the wearable device based on the first value of the torque.

A method of setting a bicycle exercise program performed by an electronic device according to an example embodiment may include setting a value of at least one parameter for a bicycle exercise program, wherein the at least one parameter is a parameter used to control a wearable device worn by a user operating based on the bicycle exercise program, when the user performs an exercise while wearing the wearable device controlled based on the value of the at least one parameter, determining a target muscle part of the user stimulated by the exercise, and outputting the target muscle part by visualizing the target muscle part differently from other muscle parts.

### BRIEF DESCRIPTION OF DRAWINGS

FIG. 1 is a diagram illustrating an overview of a wearable device worn on a body of a user according to an example embodiment.

FIG. 2 is a diagram illustrating an exercise management system including a wearable device and an electronic device according to an example embodiment.

FIG. 3 is a rear schematic view of a wearable device according to an example embodiment.

FIG. 4 is a left side view of a wearable device according to an example embodiment.

FIGS. 5A and 5B are diagrams illustrating a configuration of a control system of a wearable device according to an example embodiment.

FIG. 6 is a diagram illustrating an interaction between a wearable device and an electronic device according to an example embodiment.

FIG. 7 is a diagram illustrating a configuration of an electronic device according to an example embodiment.

FIG. 8A illustrates trajectories of a left hip joint angle and a right hip joint angle of a user walking according to an example embodiment.

FIG. 8B illustrates trajectories of a left hip joint angle and a right hip joint angle of a user pedaling a bicycle according to an example embodiment.

FIG. 9 is a flowchart illustrating a method of controlling a wearable device according to an example embodiment.

FIG. 10 is a flowchart illustrating a method of controlling a wearable device to release a torque according to an example embodiment.

FIG. 11 illustrates a method of providing a downward torque and an upward torque to a user according to an example embodiment.

FIG. 12 is a flowchart illustrating a method of setting a target joint angle based on a posture of a user according to an example embodiment.

FIG. 13 is a flowchart illustrating a method of setting a target joint angle based on a slope of a ground according to an example embodiment.

FIG. 14 is a flowchart illustrating a method of setting a value of a torque based on a slope of a ground according to an example embodiment.

FIG. 15 is a flowchart illustrating a method of determining a value of a torque provided to a joint based on a speed according to an example embodiment.

FIG. 16 is a flowchart illustrating a method of releasing an applied torque when a current state of a wearable device is an exception state according to an example embodiment.

FIG. 17 is a flowchart illustrating a method of controlling a wearable device based on a bicycle exercise program according to an example embodiment.

FIG. 18 is a flowchart illustrating a method of visualizing and outputting a muscle part based on a value of a parameter set for a bicycle exercise program according to an example embodiment.

FIG. 19 is a flowchart illustrating a method of visualizing and outputting a first pedaling section that activates a first muscle part according to an example embodiment.

FIG. 20A illustrates a method of setting a value of at least one parameter for a bicycle exercise program according to an example embodiment.

FIG. 20B illustrates a method of visualizing and outputting a first pedaling section that activates a first muscle part according to an example embodiment.

FIG. 21 illustrates a method of setting a value of at least one parameter for a bicycle exercise program based on a target muscle part received from a user according to an example embodiment.

FIG. 22 illustrates a method of setting a value of at least one parameter for each of a plurality of operating situations according to an example embodiment.

### DETAILED DESCRIPTION

Hereinafter, various embodiments of the present disclosure will be described with reference to the accompanying drawings. However, this is not intended to limit the present disclosure to specific embodiments, and it should be understood that various modifications, equivalents, and/or alternatives of the embodiments of the present disclosure are included.

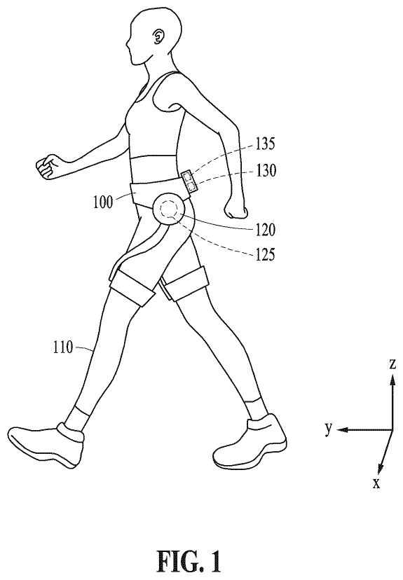

FIG. 1 is a diagram illustrating an overview of a wearable device worn on a body of a user according to an embodiment.

Referring to FIG. 1, in an embodiment, a wearable device 100 may be a device worn on a body of a user 110 to assist the user 110 in walking, exercising, and/or working. In an embodiment, the wearable device 100 may be used to measure a physical ability (e.g., a walking ability, an exercise ability, or an exercise posture) of the user 110. In embodiments, the term “wearable device” may be replaced with “wearable robot,” “walking assistance device,” or “exercise assistance device.” The user 110 may be a human or an animal, but is not limited thereto. The wearable device 100 may be worn on a body (e.g., a lower body (the legs, ankles, knees, etc.), an upper body (the torso, arms, wrists, etc.), or the waist) of the user 110 to apply an external force such as an assistance force and/or a resistance force to a body motion of the user 110. The assistance force may be a force applied in the same direction as the body motion direction of the user 110, the force to assist a body motion of the user 110. The resistance force may be a force applied in a direction opposite to the body motion direction of the user 110, the force hindering a body motion of the user 110. The term “resistance force” may also be referred to as “exercise load.”

In an embodiment, the wearable device 100 may operate in a walking assistance mode for assisting the user 110 in walking. In the walking assistance mode, the wearable device 100 may assist the user 110 in walking by applying an assistance force generated by a driving module 120 of the wearable device 100 to the body of the user 110. The wearable device 100 may enable the user 110 to walk independently or to walk for a long time by providing a force required for the user 110 to walk, thereby extending the walking ability of the user 110. The wearable device 100 may help in improving an abnormal walking habit or gait posture of a walker.

In an embodiment, the wearable device 100 may operate in an exercise assistance mode for enhancing the exercise effect of the user 110. In the exercise assistance mode, the wearable device 100 may impede a body movement of the user 110 or provide resistance to the body movement of the user 110 by applying a resistance force generated by the driving module 120 to the body of the user 110. When the wearable device 100 is a hip-type wearable device that is worn on a waist (or pelvis) and legs (e.g., thighs) of the user 110, the wearable device 100 may provide an exercise load to a leg motion of the user 110 while being worn on the legs, thereby enhancing the exercise effect on the legs of the user 110. In an embodiment, the wearable device 100 may apply an assistance force to the body of the user 110 to assist the user 110 in exercising. For example, when a person with a disability or an elderly person wants to exercise by wearing the wearable device 100, the wearable device 100 may provide an assistance force to assist a body motion during an exercise process. In an embodiment, the wearable device 100 may provide an assistance force and a resistance force in combination for each exercise section or time section, in such a manner of providing an assistance force in some exercise sections and a resistance force in other exercise sections.

In an embodiment, the wearable device 100 may operate in a physical ability measurement mode for measuring a physical ability of the user 110. The wearable device 100 may measure motion information of a user using sensors (e.g., an angle sensor 125 and an inertial measurement unit (IMU) 135) provided in the wearable device 100 while the user is walking or exercising, and evaluate the physical ability of the user based on the measured motion information. For example, a gait index or an exercise ability indicator (e.g., muscular strength, endurance, balance, or exercise motion) of the user 110 may be estimated through the motion information of the user 110 measured by the wearable device 100. The physical ability measurement mode may include an exercise posture measurement mode for measuring an exercise posture of a user.

In embodiments of the present disclosure, for convenience of description, the wearable device 100 is described as an example of a hip-type wearable device, as illustrated in FIG. 1, but the embodiments are not limited thereto. As described above, the wearable device 100 may be worn on body parts (e.g., upper arms, lower arms, hands, calves, and feet) other than the waist and legs (particularly, the thighs), and a shape and configuration of the wearable device 100 may vary depending on the body part on which the wearable device 100 is worn.

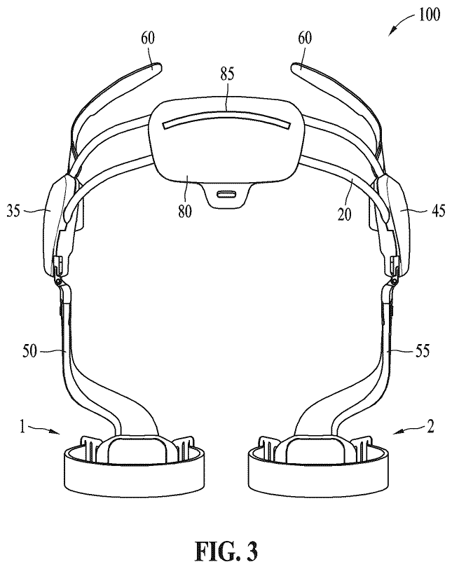

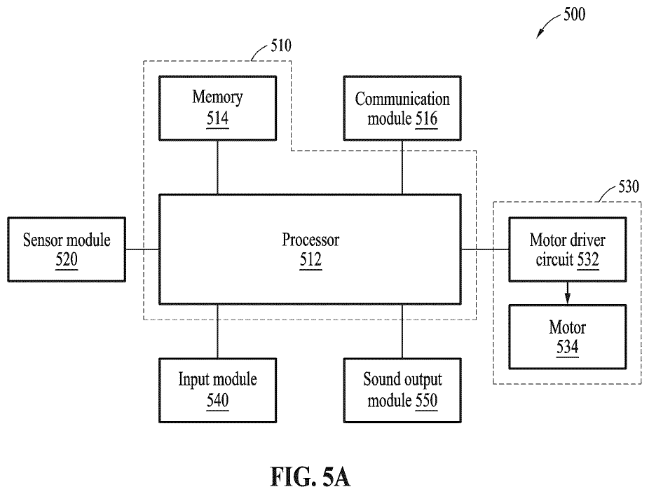

According to an embodiment, the wearable device 100 may include a support frame (e.g., leg support frames 50 and 55 and a waist support frame 20 of FIG. 3) configured to support the body of the user 110 when the wearable device 100 is worn on the body of the user 110, a sensor module (e.g., a sensor module 520 of FIG. 5A) configured to obtain sensor data including motion information on a body motion (e.g., a motion of a leg, and a motion of an upper body) of the user 110, the driving module 120 (e.g., driving modules 35 and 45 of FIG. 3) configured to generate torque to be applied to the legs of the user 110, and a control module 130 (e.g., a control module 510 of FIGS. 5A and 5B) configured to control the wearable device 100.

The sensor module may include the angle sensor 125 and the IMU 135. The angle sensor 125 may measure a rotation angle of a leg support frame of the wearable device 100 corresponding to a hip joint angle value of the user 110. The rotation angle of the leg support frame measured by the angle sensor 125 may be estimated as a hip joint angle value (or a leg angle value) of the user 110. The angle sensor 125 may include, for example, an encoder and/or a Hall sensor. In an embodiment, the angle sensor 125 may be present near each of a right hip joint and a left hip joint of the user 110. The IMU 135 may include an acceleration sensor and/or an angular velocity sensor, and may measure a change in acceleration and/or angular velocity according to a motion of the user 110. The IMU 135 may measure, for example, an upper body motion value of the user 110 corresponding to a motion value of a waist support frame (or a base body (a base body 80 of FIG. 3)) of the wearable device 100. A motion value of the waist support frame measured by the IMU 135 may be estimated as an upper body motion value of the user 110.

In an embodiment, the control module 130 and the IMU 135 may be arranged within the base body (e.g., the base body 80 of FIG. 3) of the wearable device 100. The base body may be disposed on a lumbar region (an area of the lower back) of the user 110 while the user 110 is wearing the wearable device 100. The base body may be formed or attached to an outer side of the waist support frame of the wearable device 100. The base body may be mounted on the lumbar region of the user 110 to provide a cushioning feeling to the lower back of the user 110 and may support the lower back of the user 110 together with the waist support frame.

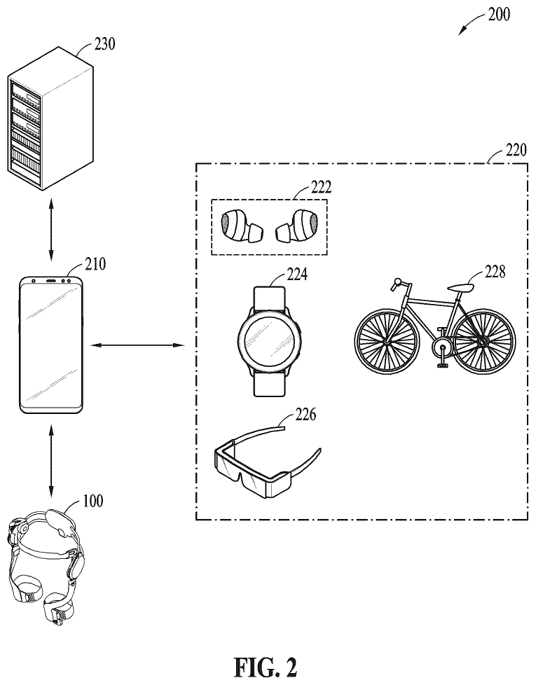

FIG. 2 is a diagram illustrating an exercise management system including a wearable device and an electronic device according to an embodiment.

Referring to FIG. 2, an exercise management system 200 may include a wearable device 100 to be worn on a body of a user, an electronic device 210, another wearable device 220, and a server 230. In an embodiment, at least one (e.g., the other wearable device 220 or the server 230) of the above devices may be omitted from the exercise management system 200, or one or more other devices (e.g., an exclusive controller device of the wearable device 100) may be added thereto.

In an embodiment, the wearable device 100 may be worn on the body of the user in a walking assistance mode to assist a motion of the user. For example, the wearable device 100 may be worn on legs of the user to help the user in walking by generating an assistance force for assisting a leg motion of the user.

In an embodiment, the wearable device 100 may generate a resistance force for hindering a body motion of the user or an assistance force for assisting a body motion of the user and apply the generated resistance force or assistance force to the body of the user to enhance the exercise effect of the user in an exercise assistance mode. In the exercise assistance mode, the user may select, through the electronic device 210, an exercise program (e.g., squat, split lunge, dumbbell squat, lunge and knee up, stretching, or the like) to perform using the wearable device 100 and/or an exercise intensity to be applied to the wearable device 100. The wearable device 100 may control a driving module of the wearable device 100 according to the exercise program selected by the user and obtain sensor data including motion information of the user through a sensor module. The wearable device 100 may adjust the strength of the resistance force or assistance force applied to the user according to the exercise intensity selected by the user. For example, the wearable device 100 may control the driving module to generate a resistance force corresponding to the exercise intensity selected by the user.

In an embodiment, the wearable device 100 may be used to measure a physical ability of the user by interworking with the electronic device 210. The wearable device 100 may operate in a physical ability measurement mode, which is a mode for measuring the physical ability of the user, under a control of the electronic device 210, and may transmit sensor data obtained by a motion of the user in the physical ability measurement mode to the electronic device 210. The electronic device 210 may estimate the physical ability of the user by analyzing the sensor data received from the wearable device 100.

The electronic device 210 may communicate with the wearable device 100 and may remotely control the wearable device 100 or provide the user with state information about a state (e.g., a booting state, a charging state, a sensing state, or an error state) of the wearable device 100. The electronic device 210 may receive sensor data obtained by a sensor of the wearable device 100 from the wearable device 100 and estimate the physical ability of the user or an exercise result based on the received sensor data. In an embodiment, when the user exercises while wearing the wearable device 100, the wearable device 100 may obtain sensor data including motion information of the user using sensors and transmit the obtained sensor data to the electronic device 210. The electronic device 210 may extract a motion value of the user from the sensor data and evaluate an exercise posture of the user based on the extracted motion value. The electronic device 210 may provide the user with an exercise posture measured value and exercise posture evaluation information related to the exercise posture of the user through a graphical user interface (GUI).

In an embodiment, the electronic device 210 may execute a program (e.g., an application) configured to control the wearable device 100, and the user may adjust an operation or a set value of the wearable device 100 (e.g., the magnitude of torque output from a driving module (e.g., driving modules 35 and 45 of FIG. 3), the volume of audio output from a sound output module (e.g., a sound output module 550 of FIGS. SA and 5B), or the brightness of a lighting unit (e.g., a lighting unit 85 of FIG. 3)) through the corresponding program. The program executed by the electronic device 210 may provide a GUI for interaction with the user. The electronic device 210 may be a device in various forms. For example, the electronic device 210 may include, but is not limited to, a portable communication device (e.g., a smartphone), a computer device, an access point, a portable multimedia device, or a home appliance device (e.g., a television, an audio device, a projector device).

According to an embodiment, the electronic device 210 may be connected to the server 230 using short-range wireless communication or cellular communication. The server 230 may receive user profile information of the user who uses the wearable device 100 from the electronic device 210 and store and manage the received user profile information. The user profile information may include, for example, information about at least one of the name, age, gender, height, weight, or body mass index (BMI). The server 230 may receive exercise history information about an exercise performed by the user from the electronic device 210 and store and manage the received exercise history information. The server 230 may provide the electronic device 210 with various exercise programs or physical ability measurement programs that may be provided to the user.

According to an embodiment, the wearable device 100 and/or the electronic device 210 may be connected to the other wearable device 220. The other wearable devices 220 may include, for example, wireless earphones 222, a smartwatch 224, smart glasses 226, or a bicycle 228, but examples are not limited to the foregoing devices. In an embodiment, the smartwatch 224 may measure a biosignal including heart rate information of the user and transmit the measured biosignal to the electronic device 210 and/or the wearable device 100. The electronic device 210 may estimate the heart rate information (e.g., a current heart rate, a maximum heart rate, and an average heart rate) of the user based on the biosignal received from the smartwatch 224 and provide the estimated heart rate information to the user.

According to an embodiment, the bicycle 228 may include at least one sensor capable of sensing a state of the bicycle 228. For example, the bicycle 228 may include a first sensor for sensing a rotational angle of an arm of a crank. For example, the bicycle 228 may include a second sensor for sensing a horizontal level of the ground on which the bicycle 228 is positioned. For example, the second sensor may be disposed on a top tube of the bicycle 228. When the user rides the bicycle 228, information generated by the first sensor and information generated by the second sensor may be transmitted to the electronic device 210 via the wearable device 100 or directly to the electronic device 210. The bicycle 228 may include a non-powered bicycle for outdoor riding, an electric bicycle, and an indoor bicycle, and are not limited thereto. For example, the bicycle 228 may be a device that allows a user to pedal.

In an embodiment, the exercise result information, physical ability information, and/or exercise posture evaluation information evaluated by the electronic device 210 may be transmitted to the other wearable device 220 and provided to the user through the other wearable device 220. State information of the wearable device 100 may also be transmitted to the other wearable device 220 and provided to the user through the other wearable device 220. In an embodiment, the wearable device 100, the electronic device 210, and the other wearable device 220 may be connected, directly or indirectly, to each other through wireless communication (e.g., Bluetooth communication or wireless-fidelity (Wi-Fi) communication).

In an embodiment, the wearable device 100 may provide (or output) feedback (e.g., visual feedback, auditory feedback, or haptic feedback) corresponding to a state of the wearable device 100 according to a control signal received from the electronic device 210. For example, the wearable device 100 may provide visual feedback through the lighting unit (e.g., the lighting unit 85 of FIG. 3) and provide auditory feedback through the sound output module (e.g., the sound output module 550 of FIGS. 5A and 5B). The wearable device 100 may include a haptic module and provide haptic feedback in the form of vibration to the body of the user through the haptic module. The electronic device 210 may also provide (or output) feedback (e.g., visual feedback, auditory feedback, or haptic feedback) corresponding to the state of the wearable device 100.

In an embodiment, the electronic device 210 may present a personalized exercise goal to the user in the exercise assistance mode. The personalized exercise goal may include respective target amounts of exercise for exercise types (e.g., strength exercise, balance exercise, and aerobic exercise) desired by the user, determined by the electronic device 210 and/or the server 230. When the server 230 determines a target amount of exercise, the server 230 may transmit information about the determined target amount of exercise to the electronic device 210. The electronic device 210 may personalize and present the target amounts of exercise for the exercise types, such as strength exercise, aerobic exercise, and balance exercise, according to a desired exercise program (e.g., squat, split lunge, or a lunge and knee up) and/or physical characteristics (e.g., the age, height, weight, and BMI) of the user. The electronic device 210 may display a GUI screen displaying the target amounts of exercise for the respective exercise types on a display.

In an embodiment, the electronic device 210 and/or the server 230 may include a database in which information about a plurality of exercise programs to be provided to the user through the wearable device 100 is stored. To achieve an exercise goal of the user, the electronic device 210 and/or the server 230 may recommend an exercise program suitable for the user. The exercise goal may include, for example, at least one of muscle strength improvement, physical strength improvement, cardiovascular endurance improvement, core stability improvement, flexibility improvement, or symmetry improvement. The electronic device 210 and/or the server 230 may store and manage the exercise program performed by the user, results of performing the exercise program, and the like.

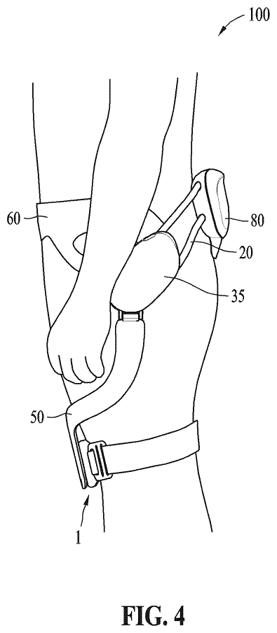

FIG. 3 is a rear schematic view of a wearable device according to an embodiment. FIG. 4 is a left side view of the wearable device according to an embodiment.

Referring to FIGS. 3 and 4, the wearable device 100 according to an embodiment may include the base body 80, the waist support frame 20, the driving modules 35 and 45, the leg support frames 50 and 55, thigh fastening portions 1 and 2, and a waist fastening portion 60. The base body 80 may include the lighting unit 85. In an embodiment, at least one (e.g., the lighting unit 85) of the above components may be omitted from the wearable device 100, or one or more other components (e.g., a haptic module) may be added to the wearable device 100.

The base body 80 may be on the waist of a user when the user wears the wearable device 100. The base body 80 worn on the waist of the user may cushion and support the waist of the user. The base body 80 may be hung on a hip region (an area of the hips) of the user such that the wearable device 100 may not be deviated downward due to gravity while the user is wearing the wearable device 100. The base body 80 may distribute a portion of a weight of the wearable device 100 to the lower back of the user while the user is wearing the wearable device 100. The base body 80 may be connected, directly or indirectly, to the waist support frame 20. Waist support frame connecting elements (not shown) to be connected, directly or indirectly, to the waist support frame 20 may be provided at both end portions of the base body 80.

In an embodiment, the lighting unit 85 may be arranged on an outer side of the base body 80. The lighting unit 85 may include a light source (e.g., a light-emitting diode (LED)). The lighting unit 85 may emit light under a control of a control module (not shown) (e.g., the control module 510 of FIGS. 5A and 5B). According to an embodiment, the control module may control the lighting unit 85 to provide (or output) visual feedback corresponding to the state of the wearable device 100 to the user through the lighting unit 85.

The waist support frame 20 may extend from both end portions of the base body 80. The lumbar region of the user may be accommodated inside the waist support frame 20. The waist support frame 20 may include at least one rigid body beam. Each beam may be in a curved shape having a preset curvature to enclose the lumbar region of the user. The waist fastening portion 60 may be connected to an end portion of the waist support frame 20. The driving modules 35 and 45 may be connected to the waist support frame 20.

In an embodiment, the control module, an IMU (not shown) (e.g., the IMU 135 of FIG. 1 or an IMU 522 of FIG. 5B), a communication module (not shown) (e.g., a communication module 516 of FIGS. 5A and 5B), and a battery (not shown) may be arranged inside the base body 80. The base body 80 may protect the control module, the IMU, the communication module, and the battery. The control module may generate a control signal for controlling an operation of the wearable device 100. The control module may include a control circuit including a processor configured to control actuators of the driving modules 35 and 45 and a memory. The control module may further include a power supply module (not shown) to supply power from a battery to each of the components of the wearable device 100.

In an embodiment, the wearable device 100 may include a sensor module (not shown) (e.g., the sensor module 520 of FIG. 5A) configured to obtain sensor data from at least one sensor. The sensor module may obtain sensor data that changes according to a motion of the user. In an embodiment, the sensor module may obtain sensor data including motion information of the user and/or motion information of the components of the wearable device 100. The sensor module may include, for example, an IMU (e.g., the IMU 135 of FIG. 1 or the IMU 522 of FIG. 5B) configured to measure an upper body motion value of the user or a motion value of the waist support frame 20, and an angle sensor (e.g., the angle sensor 125 of FIG. 1 or a first angle sensor 524 and a second angle sensor 524-1 of FIG. 5B) configured to measure a hip joint angle value of the user or a motion value of the leg support frames 50 and 55, but is not limited thereto. For example, the sensor module may further include at least one of a position sensor, a temperature sensor, a biosignal sensor, or a proximity sensor The waist fastening portion 60 may be connected to the waist support frame 20 to fasten the waist support frame 20 to a waist of the user. The waist fastening portion 60 may include, for example, a pair of belts.

The driving modules 35 and 45 may generate an external force (or torque) to be applied to the body of the user based on the control signal generated by the control module. For example, the driving modules 35 and 45 may generate an assistance force or resistance force to be applied to legs of the user. In an embodiment, the driving modules 35 and 45 may include a first driving module 45 disposed in a position corresponding to a position of a right hip joint of the user, and a second driving module 35 disposed in a position corresponding to a position of a left hip joint of the user. The first driving module 45 may include a first actuator and a first joint member, and the second driving module 35 may include a second actuator and a second joint member. The first actuator may provide power to be transmitted to the first joint member, and the second actuator may provide power to be transmitted to the second joint member. The first actuator and the second actuator may each include a motor configured to generate power (or a torque) by receiving electric power from the battery. When the motor is supplied with electric power and driven, the motor may generate a force (an assistance force) for assisting a body motion of the user or a force (a resistance force) for hindering a body motion of the user. In an embodiment, the control module may adjust the strength and direction of the force generated by the motor by adjusting the voltage and/or current supplied to the motor.

In an embodiment, the first joint member and the second joint member may receive power from the first actuator and the second actuator, respectively, and may apply an external force to the body of the user based on the received power. The first joint member and the second joint member may be arranged at positions corresponding to joint portions of the user, respectively. One side of the first joint member may be connected to the first actuator, and the other side of the first joint member may be connected to a first leg support frame 55. The first joint member may be rotated by the power received from the first actuator. An encoder or a Hall sensor that may operate as an angle sensor configured to measure the rotational angle of the first joint member (corresponding to the joint angle of the user) may be arranged on one side of the first joint member. One side of the second joint member may be connected to the second actuator, and the other side of the second joint member may be connected to a second leg support frame 50. The second joint member may rotate by the power relayed from the second actuator. An encoder or a Hall sensor that may operate as an angle sensor configured to measure a rotation angle of the second joint member may be arranged on one side of the second joint member.

In an embodiment, the first actuator may be arranged in a lateral direction of the first joint member, and the second actuator may be arranged in a lateral direction of the second joint member. A rotation axis of the first actuator and a rotation axis of the first joint member may be spaced apart from each other, and a rotation axis of the second actuator and a rotation axis of the second joint member may also be spaced apart from each other. However, embodiments are not limited thereto, and an actuator and a joint member may share a rotation axis. In an embodiment, each actuator may be spaced apart from a corresponding joint member. In this case, the driving module 35, 45 may further include a power transmission module (not shown) configured to transmit power from the actuator to the joint member. The power transmission module may be a rotary body, such as a gear, or a longitudinal member, such as a wire, a cable, a string, a spring, a belt, or a chain. However, the scope of the embodiment is not limited by a positional relationship between an actuator and a joint member and a power transmission structure described above.

In an embodiment, the leg support frame 50, 55 may support a leg (e.g., a thigh) of the user when the wearable device 100 is worn on the leg of the user. For example, the leg support frame 50, 55 may transmit power (a torque) generated by the driving module 35, 45 to the thigh of the user, and the power may function as an external force to be applied to a motion of the leg of the user. As one end portion of the leg support frame 50, 55 is connected to a joint member to rotate and the other end portion of the leg support frame 50, 55 is connected to the thigh fastening portion 1, 2, the leg support frame 50, 55 may transmit the power generated by the driving module 35, 45 to the thigh of the user while supporting the thigh of the user. For example, the leg support frame 50, 55 may push or pull the thigh of the user. The leg support frame 50, 55 may extend in a longitudinal direction of the thigh of the user. The leg support frames 50 and 55 may be bent to surround at least a portion of the circumference of the thighs of the user. The leg support frames 50 and 55 may include the first leg support frame 55 configured to support the right leg of the user and the second leg support frame 50 configured to support the left leg of the user.

The thigh fastener 1 or 2 may be connected to the leg support frame 50 or 55 and may fasten the leg support frame 50 or 55 to the thigh. The thigh fastening portions 1 and 2 may include a first thigh fastening portion 2 configured to fasten the first leg support frame 55 to a right thigh of the user, and a second thigh fastening portion 1 configured to fasten the second leg support frame 50 to a left thigh of the user.

In an embodiment, the first thigh fastening portion 2 may include a first cover, a first fastening frame, and a first strap, and the second thigh fastening portion 1 may include a second cover, a second fastening frame, and a second strap. The first cover and the second cover may apply torques generated by the driving modules 35 and 45 to the thighs of the user. The first cover and the second cover may be arranged on one sides of the thighs of the user to push or pull the thighs of the user. For example, the first cover and the second cover may be arranged on front surfaces of the thighs of the user. The first cover and the second cover may be arranged in circumferential directions of the thighs of the user. The first cover and the second cover may extend to both sides from the other end portions of the leg support frames 50 and 55 and may include curved surfaces corresponding to the thighs of the user. One ends of the first cover and the second cover may be connected to the fastening frames, and the other ends thereof may be connected to the straps.

The first fastening frame and the second fastening frame may be arranged, for example, to surround at least some portions of the circumferences of the thighs of the user, thereby preventing or reducing chances of the thighs of the user from being separated from the leg support frames 50 and 55. The first fastening frame may have a fastening structure that connects the first cover and the first strap, and the second fastening frame may have a fastening structure that connects the second cover and the second strap.

The first strap may enclose the remaining portion of the circumference of the right thigh of the user that is not covered by the first cover and the first fastening frame, and the second strap may enclose the remaining portion of the circumference of the left thigh of the user that is not covered by the second cover and the second fastening frame. The first strap and the second strap may include, for example, an elastic material (e.g., a band).

FIGS. 5A and 5B are diagrams illustrating a configuration of a control system of a wearable device according to an embodiment.

Referring to FIG. 5A, the wearable device 100 may be controlled by a control system 500. The control system 500 may include the control module 510, the communication module 516, the sensor module 520, a driving module 530, an input module 540, and the sound output module 550. In an embodiment, at least one (e.g., the sound output module 550) of the above components may be omitted from the control system 500, or one or more other components (e.g., a haptic module) may be added to the control system 500.

The driving module 530 may include a motor 534 configured to generate power (e.g., torque), and a motor driver circuit 532 to drive the motor 534. Although FIG. 5A illustrates the driving module 530 including one motor driver circuit 532 and one motor 534, the example of FIG. SA is merely an example. Referring to FIG. 5B, a control system 500-1 shown in FIG. 5B may include a plurality of (e.g., two or more) motor driver circuits 532 and 532-1 and a plurality of (e.g., two or more) motors 534 and 534-1. The driving module 530 including the motor driver circuit 532 and the motor 534 may correspond to the first driving module 45 of FIG. 3, and a driving module 530-1 including the motor driver circuit 532-1 and the motor 534-1 may correspond to the second driving module 35 of FIG. 3. The following descriptions of the motor driver circuit 532 and the motor 534 may also be respectively applicable to the motor driver circuit 532-1 and the motor 534-1 illustrated in FIG. 5B.

Referring back to FIG. 5A, the sensor module 520 may include a sensor circuit including at least one sensor. The sensor module 520 may obtain sensor data including motion information of a user or motion information of the wearable device 100. The sensor module 520 may transmit the obtained sensor data to the control module 510. The sensor module 520 may include an IMU 522 and an angle sensor (e.g., the first angle sensor 524 and the second angle sensor 524-1) as illustrated in FIG. 5B. The IMU 522 may measure an upper body motion value of the user. For example, the IMU 522 may sense X-axis, Y-axis, and Z-axis accelerations and X-axis, Y-axis, and Z-axis angular velocities according to a motion of the user. The IMU 522 may be used to measure, for example, at least one of a forward and backward tilt, a left and right tilt, or a rotation of the body of the user. In addition, the IMU 522 may obtain motion values (e.g., acceleration values and angular velocity values) of a waist support frame (e.g., the waist support frame 20 of FIG. 3) of the wearable device. The motion values of the waist support frame 20 may correspond to upper body motion values of the user.

The angle sensor may measure a hip joint angle value according to a leg motion of the user. Sensor data that may be measured by the angle sensor may include, for example, a hip joint angle value of a right leg, a hip joint angle value of a left leg, and information on a direction of a motion of a leg. For example, the first angle sensor 524 of FIG. 5B may obtain the hip joint angle value of the right leg of the user, and the second angle sensor 524-1 may obtain the hip joint angle value of the left leg of the user. The first angle sensor 524 and the second angle sensor 524-1 may each include, for example, an encoder and/or a Hall sensor. Further, the angle sensors may obtain motion values of the leg support frames of the wearable apparatus. For example, the first angle sensor 524 may obtain a motion value of the first leg support frame 55, and the second angle sensor 524-1 may obtain a motion value of the second leg support frame 50. The motion values of the leg support frames may correspond to the hip joint angle values.

In an embodiment, the sensor module 520 may further include at least one of a position sensor configured to obtain a position value of the wearable device 100, a proximity sensor configured to sense the proximity of an object, a biosignal sensor configured to detect a biosignal of the user, or a temperature sensor configured to measure an ambient temperature.

The input module 540 may receive a command or data to be used by another component (e.g., the processor 512) of the wearable device 100 from the outside (e.g., a user) of the wearable device 100. The input module 540 may include an input component circuit. The input module 540 may include, for example, a key (e.g., a button) or a touch screen.

The sound output module 550 may output a sound signal to the outside of the wearable device 100. The sound output module 550 may provide auditory feedback to the user. For example, the sound output module 550 may include a speaker configured to play back a guiding sound signal (e.g., an operation start sound, an operation error alarm, or an exercise start alarm), music content, or a guiding voice for auditorily informing predetermined information (e.g., exercise result information or exercise posture evaluation information).

In an embodiment, the control system 500 may further include a battery (not shown) configured to supply power to each component of the wearable device. The wearable device may convert the power of the battery into power suitable for an operating voltage of each component of the wearable device and supply the converted power to each component.

The driving module 530 may generate an external force to be applied to a leg of the user under the control of the control module 510. The driving module 530 may generate a torque to be applied to the legs of the user based on a control signal generated by the control module 510. The control module 510 may transmit the control signal to the motor driver circuit 532. The motor driver circuit 532 may control the operation of the motor 534 by generating a current signal (or voltage signal) corresponding to the control signal and supplying the generated current signal to the motor 534. In some cases, the current signal may not be supplied to the motor 534. When the motor 534 is supplied with the current signal and is driven, the motor 534 may generate torque for an assistance force to assist leg motion of the user or for a resistance force to impede the leg motion of the user.

The control module 510, comprising processing circuitry, may control the overall operation of the wearable device and may generate a control signal for controlling each component (e.g., the communication module 516 or the driving module 530). The control module 510 may include the processor 512 and a memory 514.

The processor 512, comprising processing circuitry, may execute, for example, software to control at least one other component (e.g., a hardware or software component) of the wearable device connected, directly or indirectly, to the processor 512, and may perform a variety of data processing or computation. The software may include an application for providing a GUI. According to an embodiment, as at least a part of data processing or computation, the processor 512 may store instructions or data received from another component (e.g., the communication module 516) in the memory 514, may process the instructions or the data stored in the memory 514, and may store result data in the memory 514. According to an embodiment, the processor 512 may include a main processor (e.g., a central processing unit (CPU) or an application processor (AP)), or an auxiliary processor (e.g., a graphics processing unit (GPU), a neural processing unit (NPU), an image signal processor (ISP), a sensor hub processor, or a communication processor (CP)) that is operable independently of or in conjunction with the main processor. The auxiliary processor may be implemented separately from the main processor or as a part of the main processor.

The memory 514 may store a variety of data used by at least one component (e.g., the processor 512) of the control module 510. The variety of data may include, for example, software, sensor data, input data or output data for instructions related thereto. The memory 514 may include a volatile memory or a non-volatile memory (e.g., random-access memory (RAM), dynamic RAM (DRAM), or static RAM (SRAM)).

The communication module 516, comprising communication circuitry, may support establishing a direct (e.g., wired) communication channel or a wireless communication channel between the control module 510 and another component of the wearable device 100 or an external electronic device (e.g., the electronic device 210 or the other wearable device 220 of FIG. 2) and performing communication via the established communication channel. The communication module 516 may include a communication circuit configured to perform a communication function. For example, the communication module 516 may receive a control signal from an electronic device (e.g., the electronic device 210) and transmit the sensor data obtained by the sensor module 520 to the electronic device. According to an embodiment, the communication module 516 may include one or more CPs (not shown) that are operable independently of the processor 512 and that support direct (e.g., wired) communication or wireless communication. According to an embodiment, the communication module 516 may include a wireless communication module (e.g., a cellular communication module, a short-range wireless communication module, or a global navigation satellite system (GNSS) communication module), and/or a wired communication module. A corresponding one of the above communication modules may communicate with another component of the wearable device 100 and/or an external electronic device via a short-range communication network, such as Bluetooth™, Wi-Fi, or infrared data association (IrDA), or a long-range communication network, such as a legacy cellular network, a 5G network, a next-generation communication network, the Internet, or a computer network (e.g., a local area network (LAN) or a wide region network (WAN)).

In an embodiment, the control system 500, 500-1 may further include a haptic module (not shown). The haptic module may provide haptic feedback to the user under the control of the processor 512. The haptic module may convert an electrical signal into a mechanical stimulus (e.g., a vibration or a movement) or an electrical stimulus which may be recognized by a user via his or her tactile sensation or kinesthetic sensation. The haptic module may include a motor, a piezoelectric element, or an electrical stimulation device. In an embodiment, the haptic module may be positioned in at least one of the base body (e.g., the base body 80), the first thigh fastening portion 2, or the second thigh fastening portion 1.

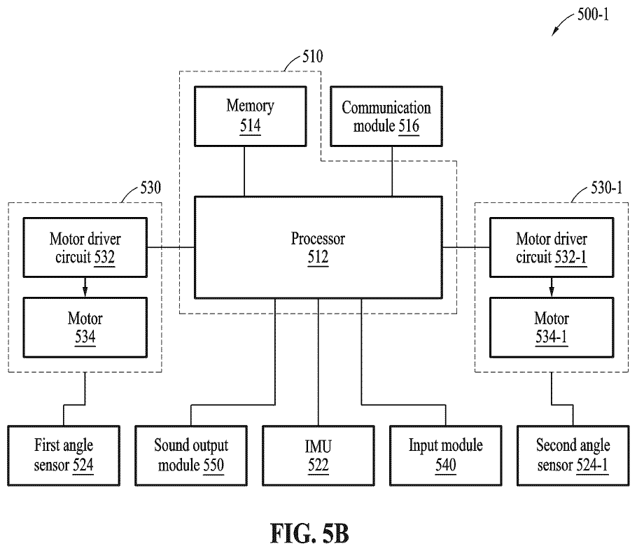

FIG. 6 is a diagram illustrating an interaction between a wearable device and an electronic device according to an embodiment.

Referring to FIG. 6, the wearable device 100 may communicate with the electronic device 210. For example, the electronic device 210 may be a user terminal of a user who uses the wearable device 100 or a controller device dedicated to the wearable device 100. In an embodiment, the wearable device 100 and the electronic device 210 may be connected to each other through short-range wireless communication (e.g., Bluetooth communication or Wi-Fi communication).

In an embodiment, the electronic device 210 may check a state of the wearable device 100 or execute an application to control or operate the wearable device 100. A screen of a user interface (UI) may be displayed to control an operation of the wearable device 100 or determine an operation mode of the wearable device 100 on a display 212 of the electronic device 210 through the execution of the application. The UI may be, for example, a GUI.

In an embodiment, the user may input an instruction for controlling the operation of the wearable device 100 (e.g., an execution instruction to a walking assistance mode, an exercise assistance mode, or a physical ability measurement mode) or change settings of the wearable device 100 through a GUI screen on the display 212 of the electronic device 210. The electronic device 210 may generate a control instruction (or control signal) corresponding to an operation control instruction or a setting change instruction input by the user and transmit the generated control instruction to the wearable device 100. The wearable device 100 may operate according to the received control instruction and transmit a control result according to the control instruction and/or sensor data measured by the sensor module of the wearable device 100 to the electronic device 210. The electronic device 210 may provide the user with result information (e.g., walking ability information, exercise ability information, or exercise posture evaluation information) derived by analyzing the control result and/or the sensor data through the GUI screen.

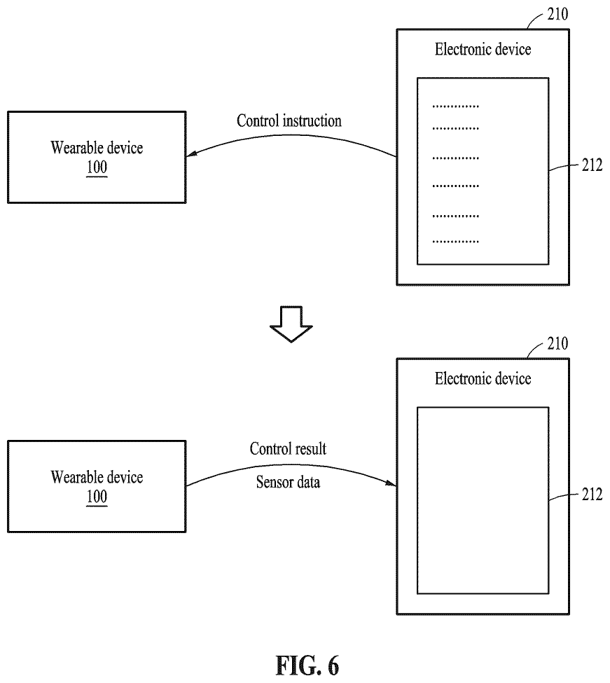

FIG. 7 is a diagram illustrating a configuration of an electronic device according to an embodiment.

Referring to FIG. 7, the electronic device 210 may include a processor 710 comprising processing circuitry, a memory 720, a communication module 730 comprising communication circuitry, a display module 740, a sound output module 750, and an input module 760. In an embodiment, at least one (e.g., the sound output module 750) of the above components may be omitted from the electronic device 210, or one or more other components (e.g., a sensor module and a battery) may be added to the electronic device 210.

The processor 710 may control at least one other component (e.g., a hardware or software component) of the electronic device 210, and may perform a variety of data processing or computation. According to an embodiment, as at least a part of data processing or computation, the processor 710 may store instructions or data received from another component (e.g., the communication module 730) in the memory 720, process the instructions or data stored in the memory 720, and store result data in the memory 720.

In an embodiment, the processor 710 may include a main processor (e.g., a CPU or an AP) or an auxiliary processor (e.g., a GPU, an NPU, an ISP, a sensor hub processor, or a CP) that is operable independently of or in conjunction with the main processor.

The memory 720 may store a variety of data used by at least one component (e.g., the processor 710 or the communication module 730) of the electronic device 210. The data may include, for example, a program (e.g., an application), and input data or output data for a command related thereto. The memory 720 may include at least one instruction executable by the processor 710. The memory 720 may include, for example, a volatile memory or a non-volatile memory.

The communication module 730 may support establishing a direct (e.g., wired) communication channel or a wireless communication channel between the electronic device 210 and another electronic device (e.g., the wearable device 100, the other wearable device 220, or the server 230) and performing communication via the established communication channel. The communication module 730 may include a communication circuit configured to perform a communication function. The communication module 730 may include one or more CPs that are operable independently of the processor 710 (e.g., an AP) and that support direct (e.g., wired) communication or wireless communication. According to an embodiment, the communication module 730 may include a wireless communication module configured to perform wireless communication (e.g., a Bluetooth communication module, a cellular communication module, a Wi-Fi communication module, or a GNSS communication module) or a wired communication module (e.g., a LAN communication module or a power line communication (PLC) module). For example, the communication module 730 may transmit a control instruction to the wearable device 100 and receive, from the wearable device 100, at least one of sensor data including body motion information of the user who is wearing the wearable device 100, state data of the wearable device 100, or control result data corresponding to the control instruction.

The display module 740 may visually provide information to the outside (e.g., a user) of the electronic device 210. The display module 740 may include, for example, a liquid-crystal display (LCD) or organic light-emitting diode (OLED) display, a hologram device, or a projector device. The display module 740 may further include a control circuit configured to control the driving of a display. In an embodiment, the display module 740 may include a touch sensor adapted to sense a touch, or a pressure sensor adapted to measure an intensity of a force incurred by the touch.

The sound output module 750 may output a sound signal to the outside of the electronic device 210. The sound output module 750 may include a guide sound signal (e.g., a driving start sound or an operation error notification sound) based on a state of the wearable device 100 and a speaker for playing musical content or a guide voice. When it is determined that the wearable device 100 is not properly worn on the body of the user, the sound output module 750 may output a guiding voice for informing the user is wearing the wearable device 100 abnormally or for guiding the user to wear the wearable device 100 normally. The sound output module 750 may output, for example, a guiding voice corresponding to exercise evaluation information or exercise result information obtained by evaluating an exercise of the user.

The input module 760 may receive a command or data to be used by another component (e.g., the processor 710) of the electronic device 210, from the outside (e.g., a user) of the electronic device 210. The input module 760 may include an input component circuit and may receive a user input. The input module 760 may include, for example, a key (e.g., a button) or a touch screen.

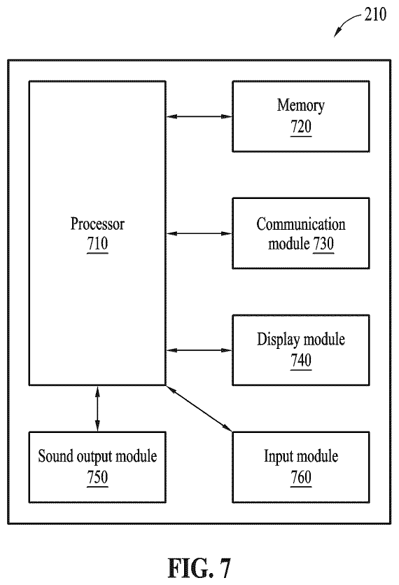

FIG. 8A illustrates trajectories of a left hip joint angle and a right hip joint angle of a user walking according to an embodiment, and FIG. 8B illustrates trajectories of a left hip joint angle and a right hip joint angle of a user pedaling a bicycle according to an embodiment.

According to an embodiment, when a person walks, trajectories of a left hip joint angle and a right hip joint angle of the person may change periodically, for example, within −40° to 30°. A point where the trajectory of the left hip joint angle and the trajectory of the right hip joint angle meet may correspond to a point where the left leg and right leg cross each other. For example, an interval having a negative sign of the hip joint angle may be an interval in which the leg is positioned in front of the torso, and an interval having a positive sign of the hip joint angle may be an interval in which the leg is positioned behind the torso.

According to an embodiment, when a user pedals a bicycle, in a state where soles of the user are positioned on the pedals, trajectories of the left hip joint angle and the right hip joint angle of the person may change periodically, for example, within −40° to 30°.

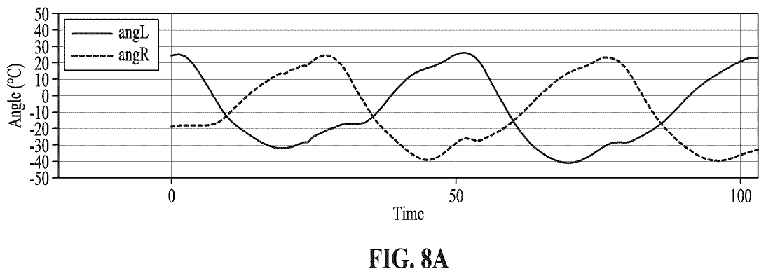

A lot of research is being conducted on methods of providing a walking assistance force or a walking resistance force to a user wearing a wearable device (e.g., the wearable device 100 of FIG. 1) through a walking program while the user is walking. Hereinafter, a method of providing an assistance force or a resistance force to a user wearing a wearable device while riding a bicycle through a bicycle exercise program will be described in detail with reference to FIGS. 9 to 22.

FIG. 9 is a flowchart illustrating a method of controlling a wearable device according to an embodiment.

Operations 910 to 950 described below may be performed by an electronic device (e.g., the wearable device 100 of FIG. 1 or the electronic device 210 of FIG. 2). The electronic device may include a processor (e.g., the processor 512 of FIG. 5A or the processor 710) and a communication module (e.g., the communication module 516 of FIG. 5A or the communication module 730).

According to an embodiment, operations 910 to 950 may be performed on each of a first joint (e.g., a left hip joint) and a second joint (e.g., a right hip joint) of a user. Although the operations performed for the first joint are described below, however, operations 910 to 950 may also be performed for the second joint.

In operation 910, the processor of the electronic device may receive first joint angle information on a first joint from a wearable device (e.g., the wearable device 100 of FIG. 1) through the communication module while the wearable device is controlled based on a bicycle exercise program. For example, the first joint may be a left hip joint or a right hip joint of the user. For example, the first joint angle information may be joint angle information generated by an angle sensor (e.g., the first angle sensor 524 or the second angle sensor 524-1 of FIG. 5B) of the wearable device.

According to an embodiment, the angle sensor of the wearable device may periodically generate the first joint angle information, and transmit the generated first joint angle information to the electronic device.

In operation 920, the processor of the electronic device may determine a first joint angle of the first joint based on the first joint angle information. For example, the first joint angle information of an encoder sensor or a Hall sensor may be raw sensor data, and the processor of the electronic device may determine the first joint angle in a range of −180° to 180° based on the sensor data.

According to an embodiment, when the first joint angle information received by the electronic device from the wearable device directly indicates the first joint angle in the range of −180° to 180°, operation 920 may not be performed.

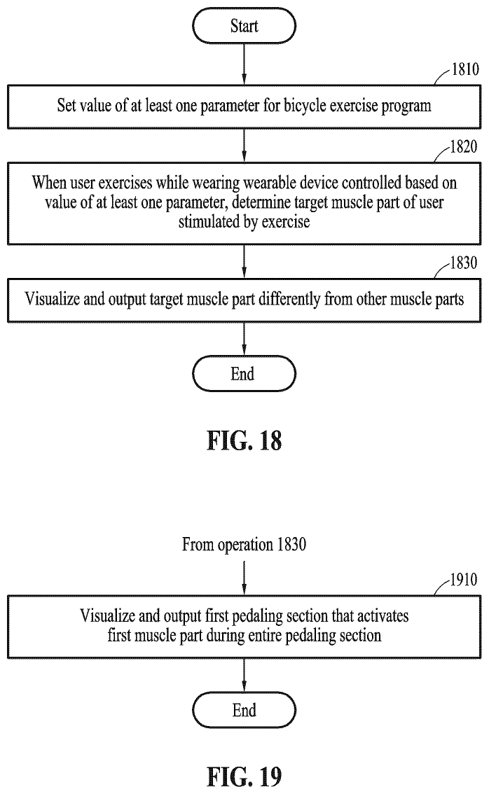

In operation 930, the processor of the electronic device may determine whether the first joint angle corresponds to a first target joint angle. For example, the first target joint angle may be a value of at least one parameter set for a bicycle exercise program being performed by the electronic device and the wearable device. For example, the at least one parameter of the bicycle exercise program may include at least one of an intensity (or a gain), a timing (or a delay), a sensitivity, a right angle offset, a left angle offset, a first target joint angle, or a second target joint angle used to determine a value of a torque to be output. A method of setting at least one parameter set for the bicycle exercise program will be described in detail below with reference to FIGS. 18 to 22.

According to an embodiment, the first target joint angle may correspond to an output point of a downward torque to be provided to the user's thighs. When the user pedals, a hip joint angle of the user at a point where the user starts pressing down on an arm of a crank may correspond to the first target joint angle. Regardless of the type of pedal on the bicycle, the downward torque may be output to the user's thighs. For example, the pedal types of a bicycle may include general pedals and locking pedals.

According to an embodiment, the first target joint angle may correspond to an output point of an upward torque to be provided to the user's thighs. When the user pedals, the hip joint angle of the user at a point where the user starts pulling the arm of the crank upward may correspond to the first target joint angle. The upward torque may be output to the user's thighs when the pedal type of the bicycle is a locking pedal. The pedal type of the bicycle the user is riding may be set as at least one parameter set for the bicycle exercise program, and when the pedal type of the bicycle is the locking pedal, the upward torque may be set to be output.

According to an embodiment, an angle of the arm of the crank of the bicycle and an angle of the hip joint of the user may be related to each other when the user's legs are placed on the pedal of the bicycle. For example, a trajectory of the arm of the crank rotating within the range of −180° to 180° and a trajectory of the hip joint angle may be related to each other.

According to an embodiment, the relationship between the angle of the arm of the crank of the bicycle and the angle of the hip joint of the user may be generated through a test walk of the bicycle. For example, the processor of the electronic device may instruct the user to place the foot on the pedal and perform a test drive. The wearable device may transmit the hip joint angle information to the electronic device while the user performs a test drive. The processor of the electronic device may generate the trajectory of the hip joint angle of the user based on the hip joint angle information. The processor of the electronic device may relate (or map) the trajectory of the hip joint angle to a rotational angle of the arm of the crank. For example, the processor of the electronic device may relate the trajectory of the hip joint angle to the rotational angle of the arm of the crank using a pre-trained model.

According to an embodiment, the electronic device may receive angle information of the arm of the crank from an external electronic device (e.g., the bicycle 228 of FIG. 2). For example, the bicycle 228 may include a sensor capable of sensing an angle of an arm of the crank, and the sensor may generate the angle information of the arm of the crank. The processor of the electronic device may receive the angle information of the arm of the crank from the bicycle 228 through the communication module. When the electronic device may directly receive the angle information of the arm of the crank, the description of the term “first joint angle information” in operations 910 to 930 may be replaced with the description of the “angle information of the arm of the crank.”

When the first joint angle does not correspond to the first target joint angle, the electronic device may control the wearable device so that no torque is applied to the user. For example, a state in which no torque is applied to the user may be a state in which a motor (e.g., the motor 534 of FIG. 5A) and a motor driver circuit (e.g., the motor driver circuit 532 of FIG. 5A) of the wearable device are not electrically connected. For example, the state may be an open state in which a + terminal and a − terminal of the motor 534 are not electrically connected to each other. In the open state, a shaft axis of the motor 534 may be connected through a leg support frame and driving gears. In the state, the user may move the user's legs freely, and the motor may not operate as a generator due to the movement of the user.

When the first joint angle corresponds to the first target joint angle, operation 940 may be performed.

In operation 940, the processor of the electronic device may determine a first value of a torque provided to the first joint (e.g., the hip joint) based on the bicycle exercise program. For example, the first value of the torque may be determined based on the value of the at least one parameter set for the bicycle exercise program. For example, the processor of the electronic device may determine the first value of the torque based on a value of the intensity (or the gain) of the torque or the sensitivity of the torque as a parameter.

According to an embodiment, the first value of the torque provided to the first joint may be determined based on an exercise mode set in the bicycle exercise program. For example, when the exercise mode is a muscular strength assistance mode, the first value of the torque may be determined to assist the movement of the first joint of the user. When the user steps on and presses down the pedal, the value of the torque that lowers the user's thigh may be determined as the first value. When the user pulls the pedal upward, the value of the torque that lifts the user's thigh upward may be determined as the first value. For example, when the exercise mode is a muscular strength strengthening mode, the first value of the torque may be determined to impede the movement of the first joint of the user. When the user steps on and presses down the pedal, the value of the torque that lifts the user's thigh upward may be determined as the first value. When the user pulls the pedal upward, the value of the torque that lowers the user's thigh may be determined as the first value.

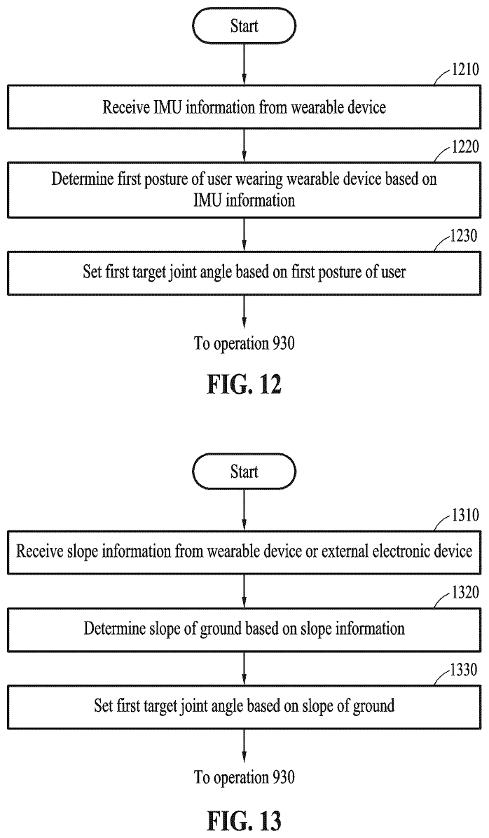

According to an embodiment, the first value of the torque may be determined based on a slope of the ground on which the bicycle is traveling. A method of determining the first value of the torque based on the slope of the ground will be described in detail below with reference to FIG. 14.

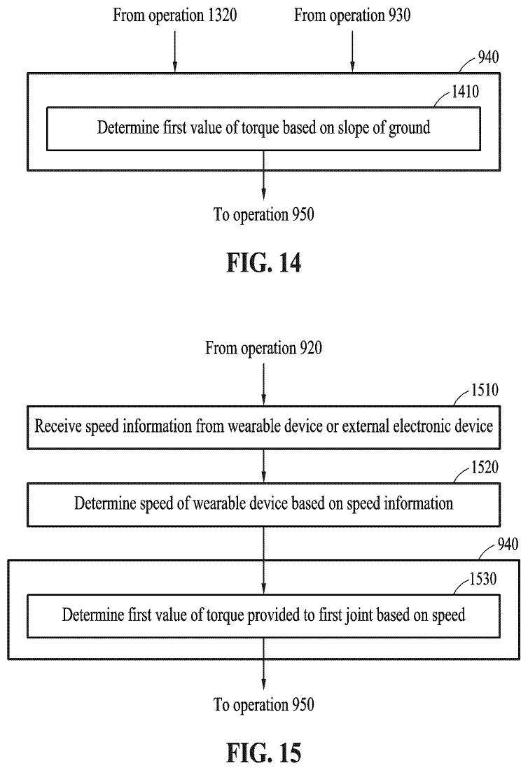

According to an embodiment, the first value of the torque may be determined based on a driving speed of the bicycle. A method of determining the first value of the torque based on the driving speed of the bicycle will be described in detail below with reference to FIG. 15.

In operation 950, the processor of the electronic device may control the wearable device based on the first value of the torque. For example, the processor of the electronic device may transmit the first value of the torque to the wearable device through the communication module, and the wearable device may control a motor driver circuit (e.g., the motor driver circuit 532 of FIG. 5A) to output the first value of the torque.

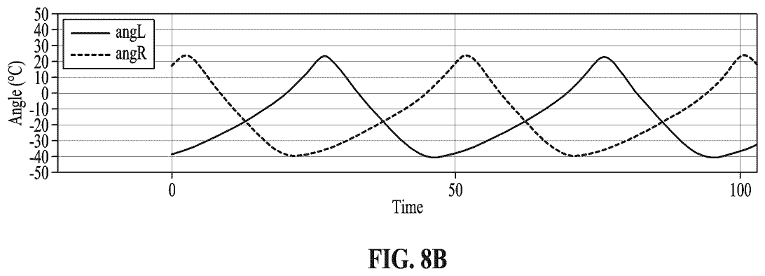

According to an embodiment, the first value of the torque may be applied to the wearable device until the torque is released. A method of releasing the torque will be described in detail below with reference to FIG. 10. For example, the first value of the torque may be a trajectory having the same value. For example, the first value of the torque may have a trajectory that changes over time. For example, the trajectory of the first value that changes over time may have a trajectory that gradually decreases

FIG. 10 is a flowchart illustrating a method of controlling a wearable device to release a torque according to an embodiment.

Operations 1010 to 1050 described below may be performed by an electronic device (e.g., the wearable device 100 of FIG. 1 or the electronic device 210 of FIG. 2). The electronic device may include a processor (e.g., the processor 512 of FIG. 5A or the processor 710) and a communication module (e.g., the communication module 516 of FIG. 5A or the communication module 730, each comprising communication circuitry). According to an embodiment, operations 1010 to 1050 may be performed after operation 950 described above with reference to FIG. 9 is performed. For example, operation 1010 may be performed in a state where the torque is being applied to the first joint of the user as a wearable device (e.g., the wearable device 100) is controlled based on the first value of the torque.

In operation 1010, the processor of the electronic device may receive second joint angle information on the first joint from the wearable device (e.g., the wearable device 100 of FIG. 1) through a communication module while the wearable device is controlled based on the bicycle exercise program. For example, the second joint angle information may be joint angle information received from the wearable device after the first joint angle information received in operation 910 described above with reference to FIG. 9.

In operation 1020, the processor of the electronic device may determine a second joint angle of the first joint based on the second joint angle information. The description of the method of determining the second joint angle of the first joint may be replaced with the description of operation 920 described above with reference to FIG. 9.

In operation 1030, the processor of the electronic device may determine whether the second joint angle corresponds to a second target joint angle. For example, the second target joint angle may be a value of the at least one parameter set for the bicycle exercise program being performed by the electronic device and the wearable device.

According to an embodiment, the second target joint angle may correspond to a release point of the downward torque provided to the user's thigh. For example, the second target joint angle for the downward torque may correspond to any point while a push motion is performed during a pedaling motion. For example, the pedaling motion may include an upper transition motion, a push motion, a lower transition, and a pull motion.

According to an embodiment, the second target joint angle may correspond to a release point of the upward torque provided to the user's thigh. For example, the second target joint angle for the upward torque may correspond to any point (e.g., an end point) while the lower transition motion is performed during the pedaling motion.

When the second joint angle corresponds to the second target joint angle, operation 1040 may be performed.

In operation 1040, the processor of the electronic device may control the wearable device to release the torque provided to the first joint of the user. For example, a state in which the torque provided to the first joint of the user is released may be a state in which a motor (e.g., the motor 534 of FIG. 5A) and a motor driver circuit (e.g., the motor driver circuit 532 of FIG. 5A) of the wearable device are not electrically connected.

According to an embodiment, after operation 1040 is performed, operation 910 described above with reference to FIG. 9 may be performed. For example, operations 910 to 950 and operations 1010 to 1040 may be performed sequentially while the user rotates the arm of the crank once.

When the pedal type is a general pedal, operations 910 to 950 and operations 1010 to 1040 for the downward torque may be performed once for one rotation of the arm of the crank. For the general pedal, a torque may be output only for the push motion through operations 910 to 950 and operations 1010 to 1040.

When the pedal type is a locking pedal, operations 910 to 950 and operations 1010 to 1040 for the downward torque may be performed once for one rotation of the arm of the crank, and operations 910 to 950 and operations 1010 to 1040 for the upward torque may be performed once. For the locking pedal, the downward torque may be output for the push motion through operations 910 to 950 and operations 1010 to 1040, and the upward torque may be output for the lower transition motion through operations 910 to 950 and operations 1010 to 1040.

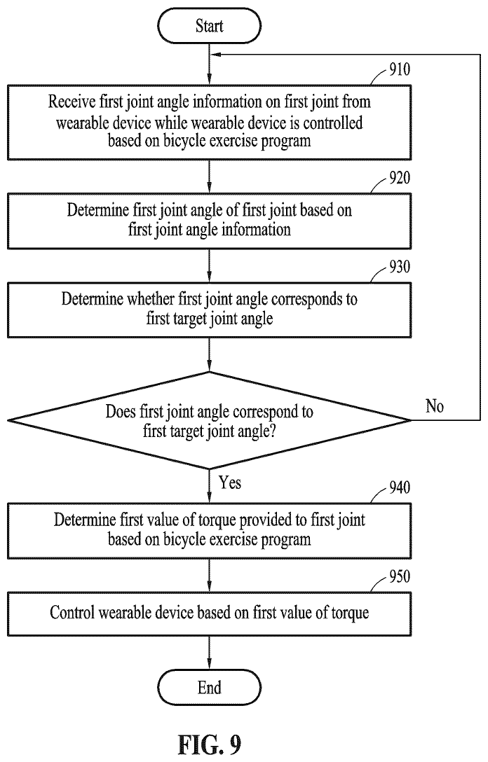

FIG. 11 illustrates a method of providing a downward torque and an upward torque to a user according to an embodiment.

According to an embodiment, a first crank arm angle 1112 and a second crank arm angle 1114 for a downward torque may be set as values of the at least one parameter set for the bicycle exercise program. The first crank arm angle 1112 and the second crank arm angle 1114 may correspond to the first target joint angle and the second target joint angle for the downward torque, respectively. In the process of setting the value of the at least one parameter of the bicycle exercise program, an electronic device (e.g., the wearable device 100 of FIG. 1 or the electronic device 210 of FIG. 2) may provide the first crank arm angle 1112 and the second crank arm angle 1114 to the user for intuitive understanding. The electronic device may relate the first crank arm angle 1112 and the second crank arm angle 1114 to the first target joint angle and the second target joint angle for the downward torque corresponding to the first crank arm angle 1112 and the second crank arm angle 1114. A crank arm angle is 0° when the arm of the crank is directed to a highest portion, and the crank arm angle may increase according to a rotation direction of the arm of the crank. The crank arm angle may be 180° when the arm of the crank is directed to a lowest portion. For example, the first crank arm angle 1112 may be 20°. For example, the second crank arm angle 1114 may be 145°.

According to an embodiment, a third crank arm angle 1122 and a fourth crank arm angle 1124 for the upward torque may be set as values of the at least one parameter set for the bicycle exercise program. The third crank arm angle 1122 and the fourth crank arm angle 1124 may correspond to the first target joint angle and the second target joint angle for the upward torque, respectively. In the process of setting the value of the at least one parameter of the bicycle exercise program, the electronic device may provide the third crank arm angle 1122 and the fourth crank arm angle 1124 to the user for intuitive understanding. The electronic device may relate the third crank arm angle 1122 and the fourth crank arm angle 1124 to the first target joint angle and the second target joint angle for the upward torque corresponding to the third crank arm angle 1122 and the fourth crank arm angle 1124. For example, the third crank arm angle 1122 may be 180°. For example, the fourth crank arm angle 1124 may be 125°.

According to an embodiment, each of the first crank arm angle 1112, the second crank arm angle 1114, the third crank arm angle 1122, and the fourth crank arm angle 1124 may be set differently depending on an exercise goal of the user. For example, the first crank arm angle 1112, the second crank arm angle 1114, the third crank arm angle 1122, and the fourth crank arm angle 1124 may each be set so that the muscle part that the user wants to stimulate is targeted while pedaling. A method of setting at least one of the first crank arm angle 1112, the second crank arm angle 1114, the third crank arm angle 1122, and the fourth crank arm angle 1124 will be described in detail below with reference to FIGS. 18 to 22.

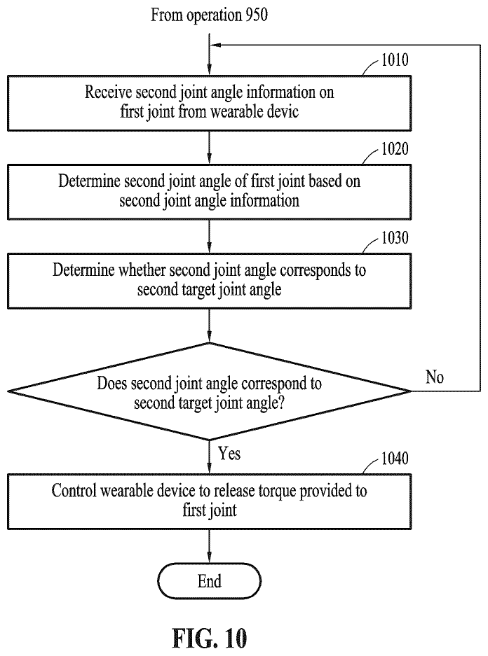

FIG. 12 is a flowchart illustrating a method of setting a target joint angle based on a posture of a user according to an embodiment.

According to an embodiment, operations 1210 to 1230 described below may be performed by an electronic device (e.g., the wearable device 100 of FIG. 1 or the electronic device 210 of FIG. 2). The electronic device may include a processor (e.g., the processor 512 of FIG. 5A or the processor 710) and a communication module (e.g., the communication module 516 of FIG. 5A or the communication module 730). According to an embodiment, operations 1210 to 1230 may be performed before operation 930 described above with reference to FIG. 9 is performed.

In operation 1210, the processor of the electronic device may receive IMU information from a wearable device (e.g., the wearable device 100 of FIG. 1) through the communication module. For example, the wearable device may generate the IMU information on a posture of a user using an IMU (e.g., the IMU 135 of FIG. 1). The wearable device may transmit the IMU information to the electronic device through the communication module. For example, the wearable device may include at least one of a waist IMU positioned on the user's waist and a thigh IMU positioned on the user's thigh. The IMU information may include at least one of the waist IMU information and the thigh IMU information.

According to an embodiment, the processor of the electronic device may receive additional IMU information from an external electronic device (e.g., the smartwatch 224, the smart glasses 226, or the bicycle 228 of FIG. 2) through the communication module. For example, the additional IMU information received from the smartwatch 224 may indicate a position of the user's wrist. For example, the additional IMU information received from the smart glasses 226 or a helmet may indicate a position of the user's head.

In operation 1220, the processor of the electronic device may determine a first posture of the user wearing the wearable device based on the IMU information. For example, the first posture of the user may be a normal posture, an aero-posture, a standing posture, or a dancing posture.

According to an embodiment, the processor of the electronic device may determine the first posture of the user based on the IMU information and the additional IMU information. For example, the processor of the electronic device may determine the relative positions of the sensors that have generated the IMU information and the additional IMU information, and determine the first posture of the user based on the relative positions of the sensors.

In operation 1230, the processor of the electronic device may set the first target joint angle based on the first posture of the user.

According to an embodiment, before the bicycle exercise program is performed, the first target joint angles for the postures of the user may be set differently as values of the at least one parameter of the bicycle exercise program. For example, the first target joint angles may be preset differently for each of the normal posture, the aero-posture, the standing posture, or the dancing posture. While the bicycle exercise program is being performed, the user may change a bicycle riding posture, and the first target joint angle may be changed and set to correspond to the changed posture. For example, when the user changes the posture from the normal posture to the dancing posture, the first target joint angle that has been set for the normal posture may be changed to the first target joint angle for the dancing posture.

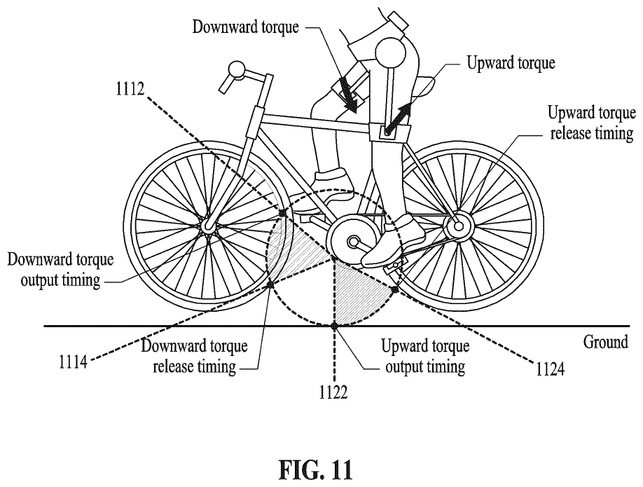

FIG. 13 is a flowchart illustrating a method of setting a target joint angle based on a slope of a ground according to an embodiment.

According to an embodiment, operations 1310 to 1330 described below may be performed by an electronic device (e.g., the wearable device 100 of FIG. 1 or the electronic device 210 of FIG. 2). The electronic device may include a processor (e.g., the processor 512 of FIG. 5A or the processor 710) and a communication module (e.g., the communication module 516 of FIG. 5A or the communication module 730). According to an embodiment, operations 1310 to 1330 may be performed before operation 930 described above with reference to FIG. 9 is performed.

In operation 1310, the processor of the electronic device may receive slope information from a wearable device (e.g., the wearable device 100 of FIG. 1) or an external electronic device through the communication module.

According to an embodiment, when a top tube of the bicycle includes a sensor for sensing horizontal level information of the bicycle, the processor of the electronic device may receive the horizontal level information as the slope information from the bicycle through the communication module.

According to an embodiment, the processor of the electronic device may receive the slope information on a location of the electronic device from a server through the communication module. For example, the electronic device may determine the location of the electronic device using a global positioning system (GPS), and receive the slope information for the determined location from the server.

In operation 1320, the processor of the electronic device may determine a slope of the ground based on the slope information. For example, the slope information may be raw sensor data, and the processor of the electronic device may determine the slope based on the sensor data.

According to an embodiment, when the slope information received in operation 1310 indicates the slope of the ground, operation 1320 may not be performed.

In operation 1330, the processor of the electronic device may set the first target joint angle based on the slope information of the ground.

According to an embodiment, before the bicycle exercise program is performed, the first target joint angle for each slope of the ground may be set differently as a value of the at least one parameter of the bicycle exercise program. The first target joint angle may be preset differently for each slope range. For example, the first target joint angle may be preset differently for each of a first slope range of 0° to 10°, a second slope range of 10° to 20°, and a third slope range of 20° to 30°. For example, when the slope changes from the first slope range to the second slope range, the first target joint angle set for the first slope range may be changed to the first target joint angle for the second slope range.

FIG. 14 is a flowchart illustrating a method of setting a value of a torque based on a slope of a ground according to an embodiment.

According to an embodiment, operation 940 described above with reference to FIG. 9 may include operation 1410 below. Operation 1410 may be performed after operation 930 described above with reference to FIG. 9 is performed and operation 1320 described above with reference to FIG. 13 is performed. For example, operation 1320 may be performed when the slope of the ground is determined. Operation 1410 may be performed by an electronic device (e.g., the wearable device 100 of FIG. 1 or the electronic device 210 of FIG. 2). The electronic device may include a processor (e.g., the processor 512 of FIG. 5A or the processor 710) and a communication module (e.g., the communication module 516 of FIG. 5A or the communication module 730).

In operation 1410, the processor of the electronic device may determine the first value of the torque based on the slope of the ground.

According to an embodiment, before the bicycle exercise program is performed, the first value of the torque for each slope of the ground may be set differently as a value of the at least one parameter of the bicycle exercise program. The first value of the torque may be preset differently for each slope range. For example, the first value of the torque may be preset differently for each of the first slope range of 0° to 10°, the second slope range of 10° to 20°, and the third slope range of 20° to 30°. For example, when the slope changes from the first slope range to the second slope range, the first value of the torque set for the first slope range may be changed to the first value of the torque for the second slope range. When the exercise mode is the muscular strength assistance mode, the first value of the torque may increase as the slope increases.

FIG. 15 is a flowchart illustrating a method of determining a value of a torque provided to a joint based on a speed according to an embodiment.

According to an embodiment, operations 1510 and 1520 below may be performed after operation 920 described above with reference to FIG. 9 is performed. Operations 1510 and 1520 may be performed by an electronic device (e.g., the wearable device 100 of FIG. 1 or the electronic device 210 of FIG. 2). The electronic device may include a processor (e.g., the processor 512 of FIG. 5A or the processor 710) and a communication module (e.g., the communication module 516 of FIG. 5A or the communication module 730).

In operation 1510, the processor of the electronic device may receive speed information (e.g., the bicycle 228 of FIG. 2) from a wearable device (e.g., the wearable device 100 of FIG. 1) or an external electronic device. For example, when the electronic device includes a GPS, the electronic device may obtain the speed information using the GPS. For example, when the electronic device includes an IMU, the electronic device may obtain IMU information as the speed information.

In operation 1520, the processor of the electronic device may determine a speed of the wearable device (or the bicycle) based on the speed information. For example, the speed information may be raw sensor data, and the processor of the electronic device may determine the speed of the wearable device based on the sensor data.

According to an embodiment, when the speed information received in operation 1510 indicates the speed of the wearable device, operation 1520 may not be performed.

According to an embodiment, operation 940 described above with reference to FIG. 9 may include operation 1530 below. Operation 1530 may be performed after operation 1520 is performed.

In operation 1530, the processor of the electronic device may determine the first value of the torque provided to the first joint based on the speed.

According to an embodiment, before the bicycle exercise program is performed, the first value of the torque for the speed of the wearable device may be set differently as a value of the at least one parameter of the bicycle exercise program. The first value of the torque may be preset differently for each speed range. For example, the first value of the torque may be preset differently for each of a first speed range of 0 km/h to 10 km/h, a second speed range of 10 km/h to 20 km/h, and a third speed range of 20 km/h to 30 km/h. For example, when the speed changes from the first speed range to the second speed range, the first value of the torque set for the first speed range may be changed to the first value of the torque for the second speed range.

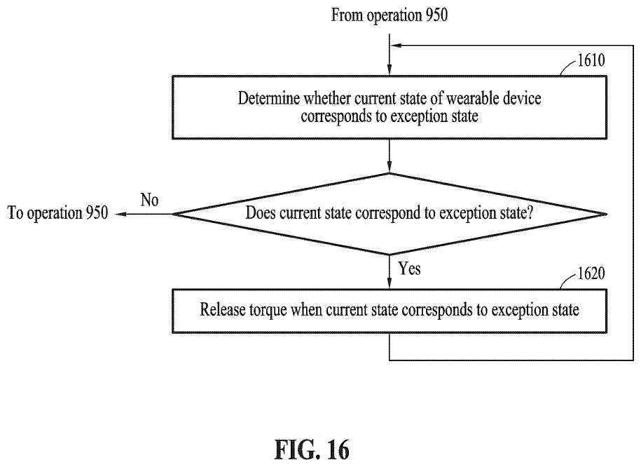

FIG. 16 is a flowchart illustrating a method of releasing an applied torque when a current state of a wearable device is an exception state according to an embodiment.

According to an embodiment, operations 1610 and 1620 below may be performed after operation 950 described above with reference to FIG. 9 is performed. Operations 1610 and 1620 may be performed by an electronic device (e.g., the wearable device 100 of FIG. 1 or the electronic device 210 of FIG. 2). The electronic device may include a processor (e.g., the processor 512 of FIG. 5A or the processor 710) and a communication module (e.g., the communication module 516 of FIG. 5A or the communication module 730).

In operation 1610, the processor of the electronic device may determine whether a current state of a wearable device (e.g., the wearable device 100 of FIG. 1) corresponds to an exception state. For example, the exception state may be a state in which it is necessary to release a torque applied to a user.

According to an embodiment, the processor of the electronic device may determine that the current state of the wearable device corresponds to the exception state when the speed of the wearable device exceeds a preset threshold speed.

According to an embodiment, the processor of the electronic device may determine that the current state of the wearable device corresponds to the exception state when the location of the wearable device (or the electronic device) is in a dangerous area. For example, the dangerous area may be a school zone or a construction zone, and is not limited to the described embodiments. The electronic device may obtain information on the dangerous area based on map data.

According to an embodiment, the processor of the electronic device may determine that the current state of the wearable device corresponds to the exception state when a dangerous terrain, such as a sharp curve or bump, appears in a travelling direction or path of the wearable device. The electronic device may obtain information on the dangerous terrain based on the map data.

According to an embodiment, the processor of the electronic device may determine whether a dangerous situation occurs in front of the user based on image information received from an external electronic device (e.g., the smart glasses 226 of FIG. 2). When the dangerous situation occurs, it may be determined that the current state of the wearable device corresponds to the exception state. For example, the dangerous situation may be a situation where the dangerous terrain such as a sharp curve or bump appears. For example, the dangerous situation may be a situation where an object appears in front of the user (e.g., a situation where a person is getting out of a parked car).

According to an embodiment, when a user input indicating an exception situation is received from the user, the processor of the electronic device may determine that the current state of the wearable device corresponds to the exception state. For example, the user may transmit the user input to the electronic device via a voice. For example, the user may transmit the user input to the electronic device by pressing a button on the wearable device or the external electronic device.

In operation 1620, the processor of the electronic device may release the torque provided to the user when it is determined that the current state of the wearable device corresponds to the exception state.

For example, a state in which no torque is applied to the user or a torque applied to the wearable device is released may be a state in which a motor (e.g., the motor 534 of FIG. 5A) and a motor driver circuit (e.g., the motor driver circuit 532 of FIG. 5A) of the wearable device are not electrically connected. For example, the state may be an open state in which a + terminal and a − terminal of the motor 534 are not electrically connected to each other. In the open state, a shaft axis of the motor 534 may be connected through a leg support frame and driving gears. In the above state, the user may move his or her legs freely, feeling only the friction from the driving gears, and may actively respond to dangerous situations.

According to an embodiment, the electronic device may output the torque based on a preset torque pattern before releasing the torque as the current state of the wearable device corresponds to the exception state. For example, the torque pattern may be preset to indicate that the torque is about to be released.

According to an embodiment, the electronic device may output a preset notification before releasing the torque as the current state of the wearable device corresponds to the exception state. For example, the notification may be a sound. For example, the notification may be output in the form of a message through a display of a wearable device or a smartwatch. For example, the notification may be a light emission from a lighting unit (e.g., the lighting unit 85 of FIG. 3) of the wearable device to notify a person behind the user of the exception state.

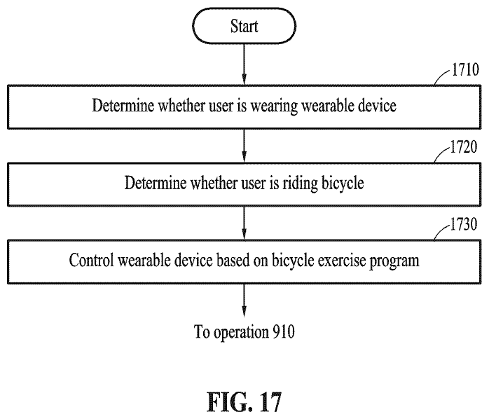

FIG. 17 is a flowchart illustrating a method of controlling a wearable device based on a bicycle exercise program according to an embodiment.

According to an embodiment, operations 1710 to 1730 described below may be performed by an electronic device (e.g., the wearable device 100 of FIG. 1 or the electronic device 210 of FIG. 2), The electronic device may include a processor (e.g., the processor 512 of FIG. 5A or the processor 710) and a communication module (e.g., the communication module 516 of FIG. 5A or the communication module 730). According to an embodiment, operations 1710 to 1730 may be performed before operation 910 described above with reference to FIG. 9 is performed.

In operation 1710, the processor of the electronic device may determine whether the user is wearing a wearable device (e.g., the wearable device 100 of FIG. 1).

According to an embodiment, the wearable device may need to be tightly attached to the body of the user to assist a motion of the user. For example, when the first thigh fastening portion 2 of the wearable device 100 is not tightly attached to the thigh of the user (e.g., the wearable device is not worn completely), malfunction of the wearable device 100 may occur. According to an embodiment, the control of the wearable device may be performed based on whether a fastening frame of the wearable device is normally connected to a cover. For example, the wearable device may determine whether the fastening frame is connected to at least a portion of a housing. The wearable device may transmit a fastening signal to the electronic device when the fastening frame is connected to the at least a portion of the housing. For example, when the wearable device is equipped with a plurality of fastening frames, the wearable device may transmit the fastening signal for each of the plurality of fastening frames to the electronic device. The electronic device may determine whether the user is wearing the wearable device based on the fastening signal received from the wearable device.

In operation 1720, the processor of the electronic device may determine whether the user is riding a bicycle.

According to an embodiment, the electronic device may determine that the user is riding a bicycle when the trajectory of the first joint angle received from the wearable device corresponds to the pedaling motion. For example, the electronic device may request pedaling through an audio output or an image output to determine the state of the user. The audio output or the image output may be performed through the electronic device or an external electronic device (e.g., the smartwatch 224 or the smart glasses 226 of FIG. 2) connected to the electronic device.

In operation 1730, the processor of the electronic device may control the wearable device based on the bicycle exercise program when it is determined that the user is riding a bicycle while wearing the wearable device. For example, the wearable device may be controlled based on the value of the at least one parameter set for the bicycle exercise program.

After operation 1730 is performed, operations 910 to 950 described above with reference to FIG. 9 may be performed.

FIG. 18 is a flowchart illustrating a method of visualizing and outputting a muscle part based on a value of a parameter set for a bicycle exercise program according to an embodiment.

Operations 1810 to 1830 described below may be performed by an electronic device (e.g., the wearable device 100 of FIG. 1 or the electronic device 210 of FIG. 2). The electronic device may include a processor (e.g., the processor 512 of FIG. 5A or the processor 710) and a communication module (e.g., the communication module 516 of FIG. 5A or the communication module 730).

According to an embodiment, operations 1810 to 1830 may be performed by an electronic device to set a user exercise program. According to an embodiment, operations 1810 to 1830 may be performed before operation 910 described above with reference to FIG. 9 is performed.

In operation 1810, the electronic device may set a value of at least one parameter for a bicycle exercise program. For example, the at least one parameter of the bicycle exercise program may include at least one of an intensity (or a gain), a timing (or a delay), a sensitivity, a right angle offset, a left angle offset, a first target joint angle, or a second target joint angle used to determine a value of a torque to be output.

According to an embodiment, the value of the at least one parameter of the bicycle exercise program may be suggested by the electronic device. For example, the electronic device may obtain an exercise goal of the user, and determine a default value preset for the exercise goal as the value of the at least one parameter. For example, the exercise goal may be a muscle part that the user wants to stimulate.

According to an embodiment, the value of the at least one parameter of the bicycle exercise program may be set (or designated) by the user. For example, the user may designate the value of the at least one parameter based on a muscle part that is visually output to be changed in response to a change in the value of the at least one parameter. For example, the electronic device may adjust the value (e.g., a default value) of the at least one parameter based on an input of the user.

According to an embodiment, the values of the at least one parameter of the bicycle exercise program may be set differently for the user's left leg and the user's right leg, respectively. For example, the first target joint angle for the user's left leg and the first target joint angle for the user's right leg may be set differently.

According to an embodiment, the electronic device may evaluate an exercise ability for each of the user's left leg and the user's right leg through the wearable device, and set the value of the at least one parameter for the user's left leg and the value of the at least one parameter for the user's right leg based on the evaluated exercise ability. For example, the electronic device may set the value of the at least one parameter for each leg for the balance of the user's left and right legs.

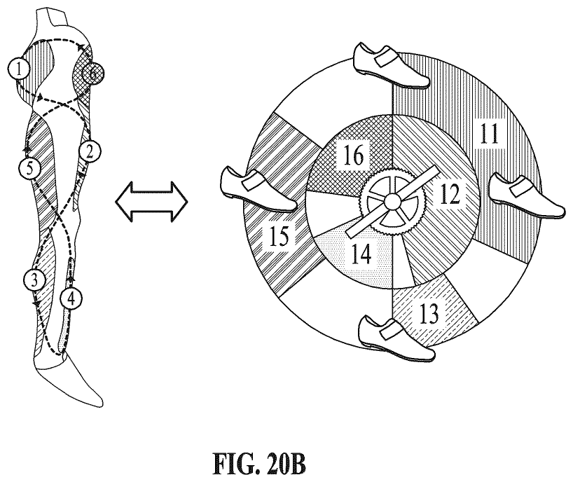

A method of setting the value of the at least one parameter of the bicycle exercise program will be described in detail below with reference to FIGS. 20A to 22.

In operation 1820, when the user performs an exercise while wearing the wearable device controlled based on the value of the at least one parameter, the electronic device may determine a target muscle part of the user stimulated by the exercise. For example, when riding a bicycle with the bicycle exercise program based on the set value of the at least one parameter, the electronic device may determine the target muscle part of the user to be stimulated.

According to an embodiment, the electronic device may determine the target muscle part corresponding to the set value of the at least one parameter using a pre-trained bicycle exercise model. For example, the pre-trained bicycle exercise model may be a neural network-based model. An input value of the bicycle exercise model may be body information of the user (e.g., a height, leg length, thigh length, calf length, age, or weight) and the value of the at least one parameter, and an output value of the bicycle exercise model may be a target muscle part.

In operation 1830, the electronic device may output the determined target muscle part by visualizing the determined target muscle part differently from other muscle parts.

When the bicycle exercise program is output or expressed only as the value of the at least one parameter, it may be difficult for the user to know what exercise effect the bicycle exercise program has. The electronic device may determine the muscle part to be stimulated by the value of the at least one parameter determined to provide the exercise effect to the user, and may visually provide the determined muscle part to the user.

According to an embodiment, the electronic device may visualize muscle parts of the lower body and output the muscle parts to the user. For example, when the determined target muscle part is a hip muscle, a hip muscle part may be output in a first color, and other muscle parts may be output in a second color.

According to an embodiment, when a confirmation input for the value of the at least one parameter is received from the user, the electronic device may finally set the value of the at least one parameter for the bicycle exercise program. The bicycle exercise program may be performed based on the value of the at least one parameter finally set for the bicycle exercise program.

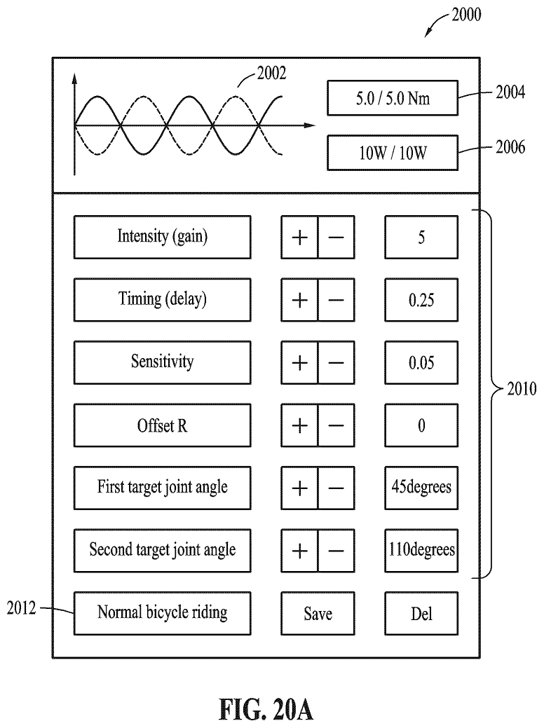

FIG. 19 is a flowchart illustrating a method of visualizing and outputting a first pedaling section that activates a first muscle part according to an embodiment.

According to an embodiment, operation 1910 below may be performed independently of and in parallel with each of operations 1810 to 1830 described above with reference to FIG. 18. Operation 1910 may be performed by an electronic device (e.g., the wearable device 100 of FIG. 1 or the electronic device 210 of FIG. 2). The electronic device may include a processor (e.g., the processor 512 of FIG. SA or the processor 710) and a communication module (e.g., the communication module 516 of FIG. 5A or the communication module 730).

In operation 1910, the electronic device may visualize and output the first pedaling section that activates the first muscle part during the entire pedaling section.

According to an embodiment, the electronic device may receive the first muscle part to be stimulated or the exercise goal from the user. For example, the electronic device may visually output muscle parts of the lower body of a person, and receive any one of the output muscle parts as the first muscle part from the user. The electronic device may determine at least one of the first target joint angle or the second target joint angle as the value of the at least one parameter for stimulating the first muscle part. The electronic device may determine at least one of the first target joint angle or the second target joint angle as the value of the at least one parameter. The electronic device may determine the first pedaling section from the entire pedaling section based on at least one of the first target joint angle or the second target joint angle, and output the determined first pedaling section by visualizing the determined first pedaling section differently from other pedaling sections. A method of visualizing and outputting the first pedaling section corresponding to the first muscle part will be described in detail below with reference to FIG. 20B.

FIG. 20A illustrates a method of setting a value of at least one parameter for a bicycle exercise program according to an embodiment.

According to an embodiment, an electronic device (e.g., the wearable device 100 of FIG. 1 or the electronic device 210 of FIG. 2) may provide the user with a UI 2000 that allows the user to adjust the value of the at least one parameter of the bicycle exercise program. For example, the UI 2000 may be an authoring tool for creating or setting up a bicycle exercise program.

According to an embodiment, the user may set a value of at least one parameter for a new bicycle exercise program by changing a value of at least one parameter set for an existing bicycle exercise program.

According to an embodiment, the user may set a value of at least one parameter of a new bicycle exercise program while generating the new bicycle exercise program. For example, the electronic device may receive an input from the user, and adjust the value of the at least one parameter based on the input of the user.

According to an embodiment, the electronic device may obtain an exercise goal from the user, and determine a default value preset for the exercise goal as the value of the at least one parameter.

For example, the UI 2000 may include a graph 2202 showing the trajectory of the first joint angle and the trajectory of the value of the torque to be output, shown in the corresponding bicycle exercise program.

For example, the UI 2000 may output a value 2006 of the torque to be output to the first joint. For example, the value 2006 of the torque may be a value of an instantaneous maximum torque or a value of a root mean square (RMS) torque.

For example, the UI 2000 may output the value 2006 of a rate represented by a force provided to the user.

For example, the UI 2000 may include a UI 2010 for adjusting the value of the at least one parameter.

For example, the UI 2000 may include a UI 2012 for designating a name of the corresponding bicycle exercise program.

FIG. 20B illustrates a method of visualizing and outputting a first pedaling section that activates a first muscle part according to an embodiment.

According to an embodiment, an electronic device (e.g., the wearable device 100 of FIG. 1 or the electronic device 210 of FIG. 2) may output a UI that visualizes and outputs a first pedaling section that activates a first muscle part. For example, the electronic device may output the UI that visualizes and outputs the first pedaling section that activates the first muscle part together with the UI 2000 described above with reference 20A.

According to an embodiment, the electronic device may visualize and output muscle parts 1, 2, 3, 4, 5, and 6 of a lower body of a person. The electronic device may distinguish and output pedaling sections 11, 12, 13, 14, 15 and 16 that activate the muscle parts 1, 2, 3, 4, 5, and 6. For example, when the user pedals in the pedaling section 11, the muscle part 1 may be activated. For example, when the user pedals in the pedaling section 12, the muscle part 2 may be activated. For example, at least a portion of the pedaling section 11 and at least a portion of the pedaling section 12 may overlap each other. The section where the pedaling section 11 and the pedaling section 12 overlap each other may be a section where the muscle part 1 and the muscle part 2 are activated simultaneously.

According to an embodiment, the electronic device may receive a first muscle part among the muscle parts 1, 2, 3, 4, 5 and 6. The first muscle part may be one or more of the muscle parts 1, 2, 3, 4, 5 and 6. For example, the first muscle part may be the muscle part 1 and the muscle part 2. The electronic device may output the muscle part 1 and the muscle part 2 as the first muscle part by visualizing the muscle part 1 and the muscle part 2 differently from the muscle parts 3, 4, 5, and 6. For example, a color of the muscle part 1 and the muscle part 2 may be output differently from a color of the muscle parts 3, 4, 5, and 6.

According to an embodiment, the electronic device may determine the first pedaling section corresponding to the first muscle part. For example, when the first muscle part is the muscle part 1 and the muscle part 2, the pedaling section 11 and the pedaling section 12 may be determined as the first pedaling section. The electronic device may output the pedaling section 11 and the pedaling section 12 by visualizing the pedaling section 11 and the pedaling section 12 differently from the pedaling sections 13, 14, 15, and 16. For example, a color of the pedaling section 11 and the pedaling section 12 may be output differently from a color of the pedaling sections 13, 14, 15, and 16.

According to an embodiment, the electronic device may set the first target joint angle and the second target joint angle based on the muscle part 1 and the muscle part 2. For example, the first target joint angle and the second target joint angle may correspond to a start angle and an end angle of the pedaling section 11 and the pedaling section 12. As in the illustrated embodiment, when there is a section where the pedaling section 11 and the pedaling section 12 overlap each other, the start angle of the pedaling section 11 may be set as the first target joint angle, and the end angle of the pedaling section 12 may be set as the second target joint angle.

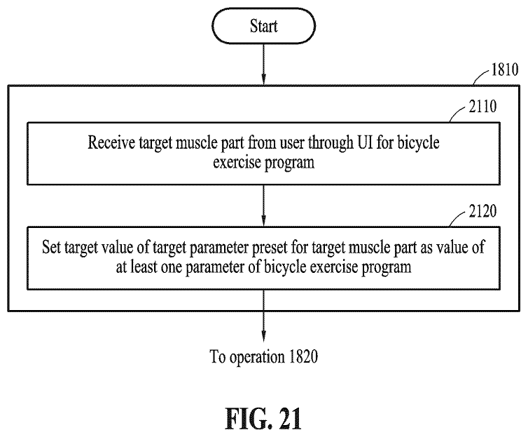

FIG. 21 illustrates a method of setting a value of at least one parameter for a bicycle exercise program based on a target muscle part received from a user according to an embodiment.

According to an example, operation 1810 described above with reference to FIG. 18 may include operations 2110 and 2120 to be described hereinafter. Operations 2110 and 2120 may be performed by an electronic device (e.g., the wearable device 100 of FIG. 1 or the electronic device 210 of FIG. 2). The electronic device may include a processor (e.g., the processor 512 of FIG. 5A or the processor 710) and a communication module (e.g., the communication module 516 of FIG. SA or the communication module 730).

In operation 2110, the processor of the electronic device may receive a target muscle part from the user through a UI for the bicycle exercise program. For example, the electronic device may receive the target muscle part from the user through a UI that visualizes and outputs the first pedaling section that activates the first muscle part described above with reference to FIG. 20B.

In operation 2120, the processor of the electronic device may set a target value of a target parameter preset for the target muscle part as the value of the at least one parameter of the bicycle exercise program. For example, the target value of the target parameter may be the first target joint angle or the second target joint angle.

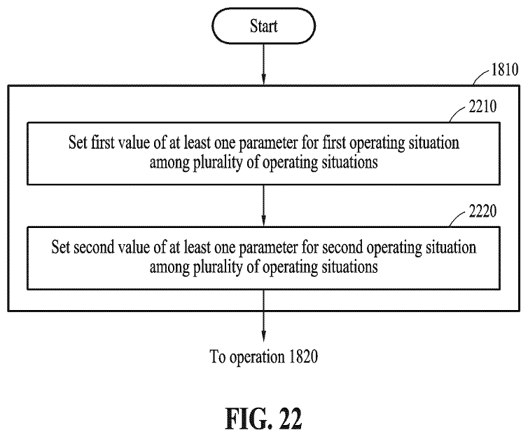

FIG. 22 illustrates a method of setting a value of at least one parameter for each of a plurality of operating situations according to an embodiment.

According to an embodiment, when the bicycle exercise program supports the plurality of operating situations, operation 1810 described above with reference to FIG. 18 may include operations 2210 and 2220 below. Operations 2210 and 2220 may be performed by an electronic device (e.g., the wearable device 100 of FIG. 1 or the electronic device 210 of FIG. 2). The electronic device may include a processor (e.g., the processor 512 of FIG. 5A or the processor 710) and a communication module (e.g., the communication module 516 of FIG. 5A or the communication module 730).

In operation 2210, the processor of the electronic device may set a first value of at least one parameter for a first operating situation among the plurality of operating situations. For example, the first operating situation may be a situation in which the bicycle exercise program is performed in the first slope range among the plurality of slope ranges of the ground. For example, the first operating situation may be a situation in which the bicycle exercise program is performed in the first speed range among the plurality of speed ranges.

In operation 2220, the processor of the electronic device may set a second value of at least one parameter for a second operating situation among the plurality of operating situations. For example, the second operating situation may be a situation in which the bicycle exercise program is performed in the second slope range among the plurality of slope ranges of the ground. For example, the second operating situation may be a situation in which the bicycle exercise program is performed in the second speed range among the plurality of speed ranges.

According to an embodiment, the first value of the parameter for the first operating situation may be the same as the second value of the parameter for the second operating situation.

According to an embodiment, the first value of the parameter for the first operating situation may be different from the second value of the parameter for the second operating situation. For example, the first value of the parameter for the first operating situation may be a value for outputting a torque that impedes the movement of the user, and the second value of the parameter for the second operating situation may be a value for outputting a torque that assists the movement of the user.

According to an embodiment, operations 2210 and 2220 may be performed when the bicycle exercise program is performed in a competitive mode. For example, the competitive mode may be a mode in which avatars corresponding to users riding bicycles in different real environments are placed in the same virtual environment, and an avatar of the user moves in the virtual environment based on exercise data of the user collected in the real environment.

For example, the different real environments may be an environment where a user rides a stationary bicycle, an environment where a user rides a bicycle on a flat ground, or an environment where a user rides a bicycle in a preset outdoor section.

For example, the virtual environment may be an environment for a virtually generated driving path. For example, the virtual environment may be an environment for an outdoor path that any one user actually drives.

According to an embodiment, the electronic device may determine an exercise level of the user based on a physical ability of the user evaluated for the competitive mode. The electronic device may determine a value of at least one parameter for the virtual environment based on the exercise level. The electronic device may determine the exercise level of the user based on a bicycle riding ability and physical strength of the user. For example, the electronic device may determine the bicycle riding ability and the physical strength of the user based on data collected through the wearable device. For example, the electronic device may determine the exercise level of the user based on a developmental level of each part of the user's body.

For example, the value of the at least one parameter for the virtual environment may be a value for implementing a virtual terrain through the wearable device. For example, when the virtual terrain is a terrain with an uphill slope, the value of the at least one parameter may be determined so that a torque that impedes the movement of the user is output. For example, when the virtual terrain is a downhill terrain, the value of the at least one parameter may be determined so that a torque that assists the movement of the user is output.

The electronic device of a first user may set, for the first user, the value of the at least one parameter for the competitive mode, and control the wearable device worn by the first user based on the value of the at least one parameter for the competitive mode, so that the users exercising in different real environments may feel as if the users are exercising in a similar environment.

According to an embodiment, a method of controlling a wearable device 100 performed by an electronic device 100; 210 may include receiving 910 first joint angle information on a first joint from the wearable device while the wearable device is controlled based on a bicycle exercise program, determining 920 a first joint angle of the first joint based on the first joint angle information, determining 930 whether the first joint angle corresponds to a first target joint angle, determining 940 a first value of a torque provided to the first joint based on the bicycle exercise program when the first joint angle corresponds to the first target joint angle, and controlling 950 the wearable device based on the first value of the torque.

According to an embodiment, the method of controlling the wearable device may include receiving second joint angle information on the first joint from the wearable device, determining a second joint angle of the first joint based on the second joint angle information, determining whether the second joint angle corresponds to a second target joint angle, and controlling 1040 the wearable device so that the torque provided to the first joint is released when the second joint angle corresponds to the second target joint angle.

According to an embodiment, the first target joint angle may be a value of at least one parameter set for the bicycle exercise program.

According to an embodiment, the determining 940 of the first value of the torque provided to the first joint may include determining the first value of the torque based on a value of at least one parameter set for the bicycle exercise program.

According to an embodiment, a value of a first parameter among the at least one parameter may be set by a user.

According to an embodiment, a value of a first parameter among the at least one parameter may be suggested by the electronic device.

According to an embodiment, the method of controlling the wearable device may further include receiving 2110 a target muscle part from a user through a user interface for the bicycle exercise program, and setting 2120 a target value of a target parameter preset for the target muscle part as a value of at least one parameter of the bicycle exercise program.

According to an embodiment, the method of controlling the wearable device may further include receiving 1210 inertial measurement unit (IMU) information from the wearable device, determining 1220 a first posture of a user wearing the wearable device based on the IMU information, and setting 1230 the first target joint angle based on the first posture of the user.

According to an embodiment, the method of controlling the wearable device may further include receiving 1310 slope information from the wearable device or an external electronic device, determining 1320 a slope of a ground based on the slope information, and setting 1330 the first target joint angle based on the slope of the ground.

According to an embodiment, the method of controlling the wearable device may further include receiving 1310 slope information from the wearable device or an external electronic device, and determining 1320 a slope of a ground based on the slope information, and the determining 940 of the first value of the torque provided to the first joint may include determining 1410 the first value of the torque based on the slope of the ground.

According to an embodiment, the method of controlling the wearable device may further include receiving 1510 speed information from the wearable device or an external electronic device, and determining 1520 a speed of the wearable device based on the speed information, and the determining 940 of the first value of the torque provided to the first joint may include determining 1530 the first value of the torque based on the speed.

According to an embodiment, the method of controlling the wearable device may further include determining 1610 whether a current state of the wearable device corresponds to an exception state, and releasing 1620 the torque when the current state corresponds to the exception state.

According to an embodiment, the method of controlling the wearable device may further include, when an exercise is performed based on a value of at least one parameter set for the bicycle exercise program, determining 1820 a target muscle part of the user stimulated by the exercise, and outputting 1830 the target muscle part by visualizing the target muscle part differently from other muscle parts.

According to an embodiment, the method of controlling the wearable device may further include visualizing and outputting 1910 a first pedaling section that activates a first muscle part during entire pedaling section of a user.

According to an embodiment, an electronic device 210 may include a communication module 730, at least one processor 710, and a memory storing instructions, and the instructions, when executed by the at least one processor 710 individually or collectively, may cause the electronic device 210 to perform receiving 910 first joint angle information on a first joint from the wearable device while the wearable device 100 is controlled based on a bicycle exercise program, determining 920 a first joint angle of the first joint based on the first joint angle information, determining 930 whether the first joint angle corresponds to a first target joint angle, determining 940 a first value of a torque provided to the first joint based on the bicycle exercise program when the first joint angle corresponds to the first target joint angle, and controlling 950 the wearable device based on the first value of the torque.

According to an embodiment, a method of setting a bicycle exercise program performed by an electronic device 210 may include setting 1810 a value of at least one parameter for a bicycle exercise program, wherein the at least one parameter is a parameter used to control a wearable device 100 worn by a user operating based on the bicycle exercise program, when the user performs an exercise while wearing the wearable device controlled based on the value of the at least one parameter, determining 1820 a target muscle part of the user stimulated by the exercise, and outputting 1830 the target muscle part by visualizing the target muscle part differently from other muscle parts.

According to an embodiment, the setting 1810 of the value of the at least one parameter for the bicycle exercise program may include obtaining an exercise goal of the user, and determining a default value preset for the exercise goal as the value of the at least one parameter.

According to an embodiment, the setting of the value of the at least one parameter for the bicycle exercise program may include adjusting the value of the at least one parameter based on an input of the user. “Based on” as used herein covers based at least on.

According to an embodiment, the method of setting the bicycle exercise program may further include visualizing and outputting 1910 a first pedaling section that activates a first muscle part during entire pedaling section.

According to an embodiment, when the bicycle exercise program supports a plurality of operating situations, the setting 1810 of the value of the at least one parameter for the bicycle exercise program may include setting 2210 a first value of the at least one parameter for a first operating situation among the plurality of operating situations, and setting 2220 a second value of the at least one parameter for a second operating situation among the plurality of operating situations.

The embodiments described herein may be implemented using a hardware component, a software component and/or a combination thereof. For example, the device, the method, and the components described in the embodiments may be implemented using a general-purpose or special-purpose computer, such as a processor, a controller, an arithmetic logic unit (ALU), a digital signal processor (DSP), a microcomputer, a field-programmable gate array (FPGA), a programmable logic unit (PLU), a microprocessor, or any other devices capable of responding to and executing instructions. The processing device may run an operating system (OS) and one or more software applications that run on the OS. The processing device also may access, store, manipulate, process, and generate data in response to execution of the software. For purpose of simplicity, the description of a processing device is used as singular; however, one skilled in the art will appreciate that a processing device may include multiple processing elements and/or multiple types of processing elements. For example, the processing device may include a plurality of processors, or a single processor and a single controller. In addition, different processing configurations are possible, such as parallel processors.

The software may include a computer program, a piece of code, an instruction, or some combinations thereof, to independently or collectively instruct or configure the processing device to operate as desired. Software and/or data may be embodied permanently or temporarily in any type of machine, component, physical or virtual equipment, computer storage medium or device, or in a propagated signal wave capable of providing instructions or data to or being interpreted by the processing device. The software may also be distributed over network-coupled computer systems so that the software is stored and executed in a distributed fashion. The software and data may be stored by one or more non-transitory computer-readable recording mediums.

The methods according to the above-described embodiments may be recorded in non-transitory computer-readable media including program instructions to implement various operations of the above-described embodiments. The media may also include, alone or in combination with the program instructions, data files, data structures, and the like. The program instructions recorded on the media may be those specially designed and constructed for the purposes of embodiments, or they may be of the kind well-known and available to those having skill in the computer software arts. Examples of non-transitory computer-readable media include magnetic media such as hard disks, floppy disks, and magnetic tape; optical media such as compact disc read-only memory (CD-ROM) discs and digital video discs (DVDs); magneto-optical media such as floptical disks; and hardware devices that are specifically configured to store and perform program instructions, such as ROM, random access memory (RAM), flash memory, and the like. Examples of program instructions include both machine code, such as produced by a compiler, and files containing higher-level code that may be executed by the computer using an interpreter.

The above-described devices may be configured to act as one or more software modules in order to perform the operations of the above-described embodiments, or vice versa.

As described above, although the embodiments have been described with reference to the limited drawings, a person skilled in the art may apply various technical modifications and variations based thereon. For example, suitable results may be achieved if the described techniques are performed in a different order and/or if components in a described system, architecture, device, or circuit are combined in a different manner and/or replaced or supplemented by other components or their equivalents.

Therefore, other implementations, other embodiments, and equivalents to the claims are also within the scope of the following claims.

1. A method of controlling a wearable device performed by an electronic device, the method comprising:
receiving first joint angle information regarding a first joint from the wearable device while the wearable device is controlled based on a bicycle exercise program;
determining a first joint angle of the first joint based on the first joint angle information;
determining whether the first joint angle corresponds to a first target joint angle;
determining a first value of a torque provided to the first joint based on the bicycle exercise program when the first joint angle corresponds to the first target joint angle; and
controlling the wearable device based on the first value of the torque.

2. The method of claim 1, further comprising:
receiving second joint angle information regarding the first joint from the wearable device;
determining a second joint angle of the first joint based on the second joint angle information;
determining whether the second joint angle corresponds to a second target joint angle; and
controlling the wearable device so that the torque provided to the first joint is released when the second joint angle corresponds to the second target joint angle.

3. The method of claim 1, wherein the first target joint angle is a value of at least one parameter set for the bicycle exercise program.

4. The method of claim 1, wherein the determining of the first value of the torque provided to the first joint comprises determining the first value of the torque based on a value of at least one parameter set for the bicycle exercise program.

5. The method of claim 3, wherein a value of a first parameter among the at least one parameter is set by a user.

6. The method of claim 3, wherein a value of a first parameter among the at least one parameter is suggested by the electronic device.

7. The method of claim 1, further comprising:
receiving a target muscle part from a user via a user interface for the bicycle exercise program; and
setting a target value of a target parameter preset for the target muscle part as a value of at least one parameter of the bicycle exercise program.

8. The method of claim 1, further comprising:
receiving inertial measurement unit (IMU) information from the wearable device;
determining a first posture of a user wearing the wearable device based on the IMU information; and
setting the first target joint angle based on the first posture of the user.

9. The method of claim 1, further comprising:
receiving slope information from the wearable device and/or an external electronic device;
determining a slope of a ground based on the slope information; and
setting the first target joint angle based on the slope of the ground.

10. The method of claim 1, further comprising:
receiving slope information from the wearable device or an external electronic device; and
determining a slope of a ground based on the slope information, wherein the determining of the first value of the torque provided to the first joint comprises determining the first value of the torque based on the slope of the ground.

11. The method of claim 1, further comprising:
receiving speed information from the wearable device and/or an external electronic device; and
determining a speed of the wearable device based on the speed information,
wherein the determining of the first value of the torque provided to the first joint comprises determining the first value of the torque based on the speed.

12. The method of claim 1, further comprising:
determining whether a current state of the wearable device corresponds to an exception state; and
releasing the torque when the current state corresponds to the exception state.

13. The method of claim 1, further comprising:
when an exercise is performed based on a value of at least one parameter set for the bicycle exercise program, determining a target muscle part of the user stimulated by the exercise; and
outputting the target muscle part at least by visualizing the target muscle part differently from other muscle parts.

14. The method of claim 1, further comprising:
visualizing and outputting a first pedaling section that activates a first muscle part during entire pedaling section of a user.

15. An electronic device comprising:
a communication module comprising communication circuitry;
at least one processor comprising processing circuitry; and
a memory storing instructions,
wherein the instructions, when executed individually and/or collectively by the at least one processor, cause the electronic device to perform:
receiving first joint angle information regarding a first joint from the wearable device while the wearable device is controlled based on a bicycle exercise program;
determining a first joint angle of the first joint based on the first joint angle information;
determining whether the first joint angle corresponds to a first target joint angle;
determining a first value of a torque provided to the first joint based on the bicycle exercise program when the first joint angle corresponds to the first target joint angle; and
controlling the wearable device based on the first value of the torque.
